(19) United States
(12) Patent Application Publication
(10) Pub. No.: US 2005/0130147 A1
(43) Pub. Date: Jun. 16, 2005

(54) Methods of preparing a targeting vector and uses thereof

(75) Inventors: Chunfang Zhang, Victoria (AU); John Morrison, Victoria (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Cpoyrat pty. Ltd., Clayton (AU)

(21) Appl. No.: 10/492,237

(22) PCT Filed: Oct. 8, 2002
(86) PCT No.: PCT/AU02/01367

(30) Foreign Application Priority Data
Oct. 9, 2001 (AU) PR 8174
May 23, 2002 (AU) PS 2522

(51) Int. Cl. C12Q 1/68; C12N 15/85

(52) U.S. Cl. 435/6; 435/455

(57) ABSTRACT

The present invention relates to providing methods for preparing a targeting construct for use in a targeting vector for gene targeting or homologous recombination. The invention also provides targeting vectors, and cells, plants and animals (including yeast) containing the vectors having predetermined modifications. The invention further provides plants and animals modified by the targeting vectors. The gene targeting methods used herein are based on transposon and recombination mediated procedures which provide for high throughput generation of deletions, which is amenable to seni automated production of knockout vectors.

The present invention relates to providing methods for preparing a targeting construct for use in a targeting vector for gene targeting or homologous recombination. The invention also provides targeting vectors, and cells, plants and animals (including yeast) containing the vectors having predetermined modifications. The invention further provides plants and animals modified by the targeting vectors.

### BACKGROUND

The integration of heterologous DNA into cells and organisms is potentially useful to produce transformed cells and organisms which are capable of expressing desired genes and/or polypeptides. However, many problems are associated with such systems. A major problem resides in the random pattern of integration of the heterologous gene into the genome of cells derived from multicellular organisms such as mammalian cells. This often results in a wide variation in the level of expression of such heterologous genes among different transformed cells. Further, random integration of heterologous DNA into the genome may disrupt endogenous genes which are necessary for the maturation, differentiation and/or viability of the cells or organism. One approach to overcome problems associated with random integration involves the use of gene targeting. This method involves the selection for homologous recombination events between DNA sequences residing in the genome of a cell or organism and newly introduced DNA sequences. This provides means for systematically altering the genome of the cell or organism.

A significant problem encountered in detecting and isolating cells, such as mammalian and plant cells, wherein homologous recombination events have occurred lies in the greater propensity for such cells to mediate non-homologous recombination.

The relative inefficiency of homologous recombination is even more problematic when working with cells that are not easily reproduced in vitro and for which the aforementioned selection and screening protocols may be impractical, if not impossible. For example, there are a large variety of cell types, including many stem cell types, which are difficult or impossible to clonally reproduce in vitro. If the relative frequency of homologous recombination itself could be improved, then it might be feasible to target a variety of cells which are not amenable to specialized isolation techniques.

Thus, there remains a significant need for gene targeting systems in which homologous recombinants can be routinely and efficiently obtained and constructed at a high enough frequency to obviate the necessity of special selection and screening protocols.

Homologous recombination is not only useful for the introduction of heterologous sequences, but this technique may also be used to eliminate, remove or inactivate sequences within the gene.

A “gene knockout” refers to the targeted inactivation of a gene in a cell or an organism. The technology relies on the replacement of the wild type gene on a chromosome (the target gene) by an inactivated gene on a targeting vector by homologous recombination. A general problem encountered in gene knockout experiments is the high frequency of random insertion of the whole vector (non-homologous recombination) in animal cells, rather than gene replacement (homologous recombination). A positive/negative selection procedure has traditionally been used to counter-select random insertion events. Generally the thymidine kinase (TK) gene from herpes simplex virus is used as the negative selection marker. While this system is routinely used, there are a number of drawbacks associated with the system. First, positive/negative selection vectors constructed are sometimes very unstable. Second, multiple cloning steps are involved in constructing a knockout vector, which takes months instead of weeks in some instances. Third, the method described herein leads itself to semi-automated approaches for the assembly of targeting vectors, which is not currently available for existing technologies.

Thus, there remains a further need for systems for the development of gene targeting vectors and for gene targeting systems in which homologous recombinants can be routinely and efficiently obtained and constructed at a high enough frequency to obviate the necessity of special selection and screening protocols.

It is an object herein to provide methods whereby stable gene targeting vectors can be constructed quickly and efficiently and for the vectors to modify any predetermined target region of the genome of a cell or organism and wherein such modified cells can be selected and enriched.

### SUMMARY OF THE INVENTION

In a first aspect of the present invention there is provided a method of preparing a targeting construct for use in a targeting vector, wherein said targeting vector is capable of modifying a target DNA sequence, said method comprising the steps of:
obtaining a copy of the target DNA sequence in vitro;
inserting a DNA sequence comprising a transposon sequence and a DNA recombination sequence at one site in the copy of the target DNA sequence;
inserting another DNA sequence comprising a transposon sequence and a DNA recombination sequence at another site in the copy of the target DNA sequence; and
inducing a recombination event between said recombination sequences to delete a portion of the copy of the target DNA sequence.

The present method described herein, provides a targeting construct that may be inserted into a targeting vector to modify target DNA sequences in the genome of cells capable of homologous recombination. This transposon-mediated procedure is particularly useful for generating deletions in target DNA sequences and cells.

In another aspect of the present invention, there is provided a pre-targeting construct for use in creating deletions in a target DNA sequence, said construct comprising:
a copy of a target DNA sequence; and
at least two transposon units each comprising a recombination sequence; and
wherein said transposon units are inserted and positioned within the copy of the target DNA so that upon recombination between the recombination sequences, a portion of the copy of target DNA is deleted.

This pre-targeting construct is a precursor of the targeting construct and exists prior to induction of recombination. This construct forms the basis of the transposon-mediated gene deletion process and is an essential component to the process. The use of the transposon units enables and facilitates deletion of DNA from the target DNA upon homologous recombination and/or from the copy of target DNA.

In another aspect of the present invention, there is provided a targeting construct prepared by the methods described herein. The targeting construct results from the recombination between the recombination sequences and lacks a portion of the copy of the target DNA and further contains DNA sequences that are homologous or substantially homologous to the target DNA. In this form the targeting construct is ideal for removal from its cloning vector and insertion into a targeting vector for use in modifying target DNA sequences by homologous recombination. The targeting construct may be isolated from the original cloning vector and reinserted and cloned into a targeting vector.

In another aspect of the present invention there is provided a double positive (DP) vector for modifying a target DNA sequence contained in the genome of a target cell capable of homologous recombination. The vector comprises a first DNA sequence which contains at least one sequence portion which is substantially homologous to a portion of a first region of a target DNA sequence. The vector also includes a second DNA sequence containing at least one sequence portion which is substantially. homologous to another portion of a second region of a target DNA sequence. A third DNA sequence is preferably positioned between the first and second DNA sequences and encodes a positive selection marker which when expressed is functional in the target cell in which the vector is used.

Within the third DNA sequence, there is provided another DNA sequence that supports high-efficiency DNA recombination in the presence of a site-specific recombinase. A suitable sequence is a loxP sequence or a FRT sequence, which is under the influence of a site specific recombinase such as Cre or FLP respectively.

A fourth DNA sequence that is also a sequence that supports high-efficiency DNA recombination in the presence of a site-specific recombinase and is functional in the target cell, is positioned 5′ to the first and/or 3′ to the second DNA sequence and is substantially incapable of homologous recombination with the target DNA sequence. This sequence may also be another loxP site or FRT under the influence of a site-specific recombinase such as Cre or FLP.

Preferably, an additional DNA sequence which acts as a primer may be added either at the 5′ or 3′ ends of the vector or is 5′ or 3′ to the first or second DNA sequence respectively.

The above DP vector containing two homologous portions and a positive selection marker can be used in the methods of the invention to modify target DNA sequences. In this method, cells are first transfected with the above vector. During this transformation, the DP vector is most frequently randomly integrated into the genome of the cell. In this case, substantially all of the DP vector containing the first, second, third and fourth DNA sequences is inserted into the genome. However, some of the DP vector is integrated into the genome via homologous recombination. When homologous recombination occurs between the homologous portions of the first and second DNA sequences of the DP vector and the corresponding homologous portions of the endogenous target DNA of the cell, the fourth DNA sequence containing the sequence that supports high-efficiency DNA recombination in the presence of a site specific recombinase is not incorporated into the genome. This is because the sequence lies outside of the regions of homology in the endogenous target DNA sequence.

As a consequence, at least two cell populations are formed. There is a cell population wherein random integration of the vector has occurred which can be selected for by way of the sequence that supports high-efficiency DNA recombination in the presence of a site specific recombinase contained in the fourth DNA sequence. This is because random events occur by integration at the ends of linear DNA. The other cell population wherein gene targeting has occurred by homologous recombination are positively selected by way of the positive selection marker contained in the third DNA sequence of the vector. Activation of a recombinase such as Cre can deactivate the positive selection marker if the fourth DNA sequence is present by deactivating that portion of the vector flanked preferably by the loxP sequences. This cell population does not contain the positive selection marker and thus does not survive the positive selection. The net effect of this positive selection method is to substantially enrich for transformed cells containing a modified target DNA sequence and hence homologous recombination.

If in the above DP vector, the third DNA sequence containing the positive selection marker is positioned between first and second DNA sequences corresponding to DNA sequences encoding a portion of a polypeptide (e.g. within the exon of a eukaryotic organism) or within a regulatory region necessary for gene expression, homologous recombination allows for the selection of cells wherein the gene containing such target DNA sequences is modified such that it is non-functional.

If, however, the positive selection marker contained in the third DNA sequence of the DP vector is positioned within an untranslated region of the genome, (e.g. within an intron in a eukaryotic gene), modifications of the surrounding target sequence (e.g. exons and/or regulatory regions) by way of substitution, insertion and/or deletion of one or more nucleotides may be made without eliminating the functional character of the target gene.

The invention also includes transformed cells containing at least one predetermined modification of a target DNA sequence contained in the genome of the cell.

In addition, the invention includes organisms such as non-human transgenic animals and plants which contain cells having predetermined modifications of a target DNA sequence in the genome of the organism.

Various other aspects of the invention will be apparent from the following detailed description, appended figures and claims.

### FIGURES

FIG. 1 shows the construction of mini-Mu transposons and their use in the generation of deletions. Mini-Mu transposon-1 may be constructed containing a double (bacterial and eukaryotic) promoter (P/P) separated by a LoxP site (open triangle) from a chloramphenicol resistance gene (Cam^{r}), the whole structure being flanked by transposon ends. In this transposon, both the bacterial promoter and the eukaryotic promoter can drive the expression of the Cam^{r} gene. Mini-Mu transposon-2 may be constructed containing the following: the tetracycline resistance gene (Tet^{r} with a bacterial promoter)—a LoxP sequence—a promoter-less β-geo gene, the whole structure being flanked by transposon ends.

Structure of mini-Mu transposons and their use in the generation of deletions. P/P, prokaryotic/eukaryotic double promoter; triangle, LoxP sequence; Cam^{r}, chloramphenicol resistant gene withour promoter; Tet^{r}, tetracycline resistant gene with its native bacterial promoter; β-geo, neomycin resistance-LacZ fusion gene without promoter; thick line, cloned target gene; thine line, vector backbone.

FIG. 2 shows a flowchart showing the procedure for the generation of deletion in a cloned animal gene.

FIG. 3 shows the principle of the DP vector. A. The design of the double positive vector. The triangles represent LoxP recombination sites which are activated by the recombinase: Cre. B. After expression of the Cre recombinase, the cells with a gene-targeted event will be still resistant to neomycin (the second positive selection). C. The cells with a random insertion will become sensitive to neomycin due to deletion between any two LoxP sites, which would eliminate either the promoter or the structural neo^{r} gene, or even both. D. Example of the approach that could be taken to construct a DP vector system.

FIG. 4 shows an alternate DP vector with two positive selectable markers. Triangles indicate loxP sites.

FIG. 5 shows the construction of the vector pCO10 carrying mini-mu transposon 1.

FIG. 6 shows testing of mini-mu transposon 1.

FIG. 7 shows the construction of the vector pCO20 carrying transposon. Mu2-Neo.

FIG. 8 shows the construction of vector pCO25 carrying transposon Mu2-HygEGFP.

FIG. 9 shows the construction of vector pCO43 carrying transposon Mu2-β-geo.

FIG. 10 shows a 24 kb Xhol fragment containing part of exon 3 and exons 4-9 of the rat HPRT gene and the mini-mu transposon 1 insertions on this cloned fragment.

FIG. 11 shows the DP vector with a promoter driving the expression of Neo^{r}.

FIG. 12 shows testing of the DP vector.

FIG. 13 shows a DP vector with two positive selectable markers.

FIG. 14 shows these targeting constructs for knockout vectors for the rat HPRT gene.

FIG. 15 shows the construction of a vector with floxed β-geo.

FIG. 16 shows a design of a southern strategy to verify HPRT knockouts.

### DESCRIPTION OF THE INVENTION

In a first aspect of the present invention there is provided a method of preparing a targeting construct for use in a targeting vector, wherein said targeting vector is capable of modifying a target DNA sequence, said method comprising the steps of:
obtaining a copy of the target DNA sequence in vitro;
inserting a DNA sequence comprising a transposon sequence and a DNA recombination sequence at one site in the copy of the target DNA sequence;
inserting another DNA sequence comprising a transposon sequence and a DNA recombination sequence at another site in the copy of the target DNA sequence; and
inducing a recombination event between said recombination sequences to delete a portion of the copy of the target DNA sequence.

The present method described herein, provides a targeting construct that may be inserted into a targeting vector to modify target DNA sequences in the genome of cells capable of homologous recombination. This transposon-mediated procedure is particularly useful for generating deletions in target DNA sequences and cells.

The recombination event converts the copy of the target DNA into a targeting construct comprising homologous or substantially homologous portions of the target DNA but having a portion of the target DNA deleted from the targeting construct. Essentially two transposons, each containing a recombination sequence may be inserted in the copy of the target gene at different locations. Recombination between the recombination sequences on the two transposons will enable the deletion of the sequence between the transposons. It is preferred that a selectable marker is left at the deletion site for positive selection in animal cells. This will be particularly useful if various deletions in a given gene are desired, facilitating the high throughput generation of deletions in cloned genes.

Throughout the description and claims of this specification, the word “comprise” and variations of the word, such as “comprising” and “comprises”, is not intended to exclude other additives, components, integers or steps.

As used herein, a “target DNA sequence” is a predetermined region within the genome of a cell which is targeted for modification by the targeting vectors of the invention. Target DNA sequences includes all structural components of genes (i.e., DNA sequences encoding polypeptides including in the case of eukaryotes, introns and exons), regulatory sequences such as enhancers sequences, promoters and the like and other regions within the genome of interest. A target DNA sequence may also be a sequence which, when targeted by a vector has no effect on the function of the host genome. Each target DNA sequence contains a homologous sequence portion which is used to design the targeting vector(s) of the invention.

The term “copy of the target DNA sequence” as used herein includes homologous copies, substantially homologous copies or modified copies of the target DNA. Homologous copies of the target DNA are identical to the target and are particularly useful where a clear deletion of a portion of the target DNA is desired. “Substantially homologous” copies are those DNA sequences that are nearly identical but will still hybridise under stringent conditions. “Modified” copies are those sequences that are “substantially homologous” but includes a change or modification that is desired for insertion into the targeting vector and ultimately into the genome of the cell. These DNA sequences will become modifying DNA sequences for use in the targeting vector. In the modified DNA, it is preferred that the modification be located outside of the sites of the recombination sequences such that upon recombination, these modified sequences remain in the targeting construct.

The copy of target DNA sequence may be obtained from any source and are generally obtained from commercial libraries. Generally, the target DNA sequences are known sequences or known genes. However, the invention is not limited to known sequences. Portions of DNA can be identified for modification and providing a copy of that portion can be made either by extraction, naturally, or by synthetic methods, it is within the scope of this invention. For the purposes of this invention, the sequences are used in vitro for manipulation and deletion of desired sequences. They may be cloned in any vector or cloning vector that enables a stable construct comprising the target DNA and for receiving the inserted DNA sequences comprising transposon and recombination sequences. Preferably, the construct is any available construct obtained through commercial sources. However, the vector pNEB193 has been found to be useful. Others include pUC based vectors in general, cosmid-based, PAC/BAC based or YAC based vectors.

The targeting constructs are so constructed for insertion into targeting vectors. The method described herein enables efficient removal of a portion of the copy of the target DNA sequence in vitro which represents the desired deletion in a target animal gene. This convenient procedure markedly simplifies the procedures for generating deletions in a target cell. It greatly reduces the need to create gene constructs which require accurate placement of DNA inserts including promoter sequences, selection markers and homologous target sequences in targeting vectors to ensure accurate homologous recombinations.

The targeting constructs include at least two separate inserted DNA sequences comprising a transposon sequence and a DNA recombination sequence. However, multiple inserted DNA sequences may be used to induce the recombination events to delete a portion of the copy of the target DNA sequence.

The transposon sequences are discrete DNA segments able to insert into new sites on DNA molecules, a process referred to as transposition. Such elements are present in both prokaryotes and eukaryotes. In bacteria, there exist several classes of such elements containing many different transposons, some of which have been studied in detail and used to insert into eukaryotic genes as well as bacterial genes for the purpose of insertional mutagenesis. Transposons have also been used as portable markers for cloning and as a tool to insert primer binding sites for sequencing.

The mechanisms and modes of transposition vary for different transposons. Generally, transposition involves the recognition of the transposon ends by the transposon-encoded transposase followed by recombination events between the transposon ends and the target site. Most transposase genes function in cis, catalysing the transposition of transposons on the same DNA molecules containing the transposase genes. Often, accessory protein and DNA cofactors are required for in vivo transposition and some transposons confer transposition immunity, preventing the transposition of the same transposons to the DNA molecules containing them.

A number of in vitro transposon systems have been developed, where only two components are required, a mini-transposon or mini-mu transposon containing an antibiotic resistance marker flanked by the ends of various transposons and a purified transposase catalysing the transposition of a donor transposon to a recipient DNA molecule. In such systems, generally, the transposase alone is sufficient and no accessory protein and DNA cofactors are required. Furthermore, there is no transposition immunity and more than one transposon can be inserted to the same DNA molecule.

Suitable mini-transposons may be selected from the group including Mu1-Cam, Mu2-Neo, Mu2-Hyg EGFP and Mu2-β-geo.

The inserted DNA sequences further include a recombination sequence. The recombination sequences as described herein are important in the process of deletion of the portion of the copy of the target DNA sequences or for any sequences that the recombination sequences flank. These sequences support high efficient recombination in the presence of a site specific recombinase.

A suitable sequence includes those of the loxP or inverted repeat sequences (FRTs) which are under the influence of recombinases such as Cre or FLP respectively. Other members of the Intergrase family of recombinases (Gln, Hin, resolvase) are also included in this description. Other enzymatically mediated or high efficiency recombination events can potentially mediate this system.

The Cre-Lox P recombinase system is most preferred. However, the FLP-FRT system may also be used. The Lox P is a 34 bp stretch of DNA which recombines with another Lox P sequence where the process is mediated by a recombinase: Cre. The recombination event cyclizes the DNA and causes the deletion of the DNA sequence between the Lox P sites. One important feature of this system is that recombination between the Lox P sequences in the same orientation will delete the DNA between the sequences, leaving one copy of Lox P. The system functions in both prokaryotes and in higher organisms and hence this method described herein is useful for all cell types including yeasts.

The recombination sequences of the inserted DNA-sequences must be compatible in so far as they must be able to recombine. Therefore, for example, if the recombination sequence of one DNA sequence is a Lox P sequence, then the other DNA sequence must include a Lox P sequence thereby facilitating the removal or deletion of sequences that they flank.

Although preferred, the transposon sequence may also contain other DNA sequences providing selectable markers and promoter sequences. These DNA sequences provide means to identify successful transposon insertion into the copied target DNA sequence. Any selectable markers may be used in combination with the transposon sequence.

Suitable markers include antibiotic resistance markers or enzyme based markers such as chloramphenicol, tetracycline, or neomycin resistance markers or the β-geo marker.

The DNA sequences that are inserted into the copy of the target DNA sequence may be inserted to ensure that the transposons are in the same orientation, thereby ensuring the recombination sequences are also in the same orientation.

The positioning of the recombination sequence within the inserted DNA sequence may be positioned in any spatial order relative to the transposon or additional selection markers and promoters providing they flank sequences that are targeted for deletion. The recombination sequences may be advantageously placed to activate new marker sequences upon deletion of a portion of the copy of the target DNA sequence.

Without being limited by theory, one inserted DNA sequence may comprise transposon sequences flanking a promoter sequence which drives a first selectable marker such as Cam^{r} and a recombination sequence is inserted between the promoter sequence and the selectable marker. Another inserted DNA sequence may comprise transposon sequences flanking an active selectable marker such as Tet^{r} and a non-active selectable marker such as β-geo and a recombination sequence is inserted between the active and non-active selectable markers. Activation of a recombination event between the recombination sequences causes a deletion of the first selectable marker and the active selectable marker resulting in activation of the non-active marker for further identification of successful recombination.

The inserted DNA sequences may be inserted into the copy of the target DNA by the process of transposition and recognition of transposon ends in the DNA sequence by a transposon encoded transposase. Ideally, each DNA sequence is inserted separately to ensure sequential integration into the copy of the target DNA. Successful integration may be identified by selection markers. However, the inserted DNA sequences may be inserted simultaneously. Although not ideal, this method may be employed providing matching recombination sequences are introduced into the copy of the target DNA and that they flank the portion of DNA that is intended for deletion.

Integration of the transposon is random. Determination of site and orientation of transposons may be achieved by PCR screening or by the use of restriction digest mapping.

Induction of a recombination event between the recombination sequences may be achieved preferably by introducing Cre into the cell.

The cre recombinase gene may be expressed, preferably by controlled means. The Tet-on system or the ecdysine inducible system can be used to induce cre expression. These systems require the integration of two plasmids into the chromosome. In transgenic and knockout experiments, chromosomally integrated plasmid DNA may cause undesirable side effects. To overcome this problem, transient expression of the Cre recombinase is most desirable to remove DNA segments flanked by LoxP sites. The adenovirus vectors are preferred for use in transient expression of the recombinase. These vectors rarely integrate into the chromosome and they do not replicate in normal cell lines, because they are replication-defective and can only be propagated in special cell lines providing the necessary replication functions. Furthermore, the transfection efficiency is much higher than plasmid expression vectors (approaching 100% for adenoviruses compared with ˜20% for plasmid expression vectors). Adenovirus vectors for transient expression of the Cre gene are most preferred. The Adenovirus expressing the Cre recombinase AxCANCre (RIKEN, Japan) is preferred. An anti-Cre antibody (Novagen) may be used to confirm the expression of the Cre recombinase by Western blotting.

Where other recombination sequences are used such as the FLP-FRT system or other members of the integrase family of recombinases the corresponding recombinases may be induced in a similar manner.

In another aspect of the present invention, there is provided a pre-targeting construct for use in creating deletions in a target DNA sequence, said construct comprising:
a copy of a target DNA sequence; and
at least two transposon units each comprising a recombination sequence; and
wherein said transposon units are inserted and positioned within the copy of the target DNA so that upon recombination between the recombination sequences, a portion of the copy of target DNA is deleted.

This pre-targeting construct is a precursor of the targeting construct and exists prior to induction of recombination. This construct forms the basis of the transposon-mediated gene deletion process and is an essential component to the process. The use of the transposon units enables and facilitates deletion of DNA from the target DNA upon homologous recombination and/or from the copy of target DNA.

In a preferred embodiment, the transposon unit is a mini-mu transposon unit. The mini-mu transposon unit further comprises a selection marker and desirably a promoter for expression of the selection marker. The transposon units are preferably different so that upon recombination, a different selection marker or gene is activated to facilitate selection of recombinants and targeting constructs.

The choice of selection markers is as previous described.

In another aspect of the present invention, there is provided a targeting construct prepared by the methods described herein. The targeting construct results from the recombination between the recombination sequences and lacks a portion of the copy of the target DNA and further contains DNA sequences that are homologous or substantially homologous to the target DNA. In this form the targeting construct is ideal for removal from its cloning vector and insertion into a targeting vector for use in modifying target DNA sequences by homologous recombination. The targeting construct may be isolated from the original cloning vector and reinserted and cloned into a targeting vector.

A preferred form of the targeting construct includes an active selection marker so formed following a recombination event and wherein the active selection marker results from a combination of promoter and selection markers deriving separately from the inserted DNA sequences. Preferably the active selection marker is flanked by DNA sequences that are homologous or substantially homologous to the target DNA. This selection marker aids in positive selection both in the cloning vector and in cells that will have used the targeting vector in which the targeting construct is deployed.

This transposon mediated procedure has been designed to generate deletions and to speed up vector construction as soon as a gene is cloned. Because the transposon insertion on the cloned gene is random and there is no sequence preference for the mini-Mu transposons, deletions may be generated at any desired positions without further cloning steps for each deletion. This facilitates high throughput generation of deletions, which is potentially amenable to a semi-automated production of knockout vectors.

A preferred targeting vector is a DP (Double Positive) vector as herein described.

In another aspect of the present invention there is provided a double positive (DP) vector for modifying a target DNA sequence contained in the genome of a cell, said DP vector comprising:
a first homologous vector DNA sequence capable of homologous recombination with a first region of said target DNA sequence;
a positive selection marker DNA sequence capable of conferring a positive selection characteristic in said cells;
a third sequence that supports high-efficiency DNA recombination in the presence of a site specific recombinase and which is contained within the positive selection marker;
a second homologous vector DNA sequence capable of homologous recombination with a second region of said target DNA sequence; and
a fourth sequence which directs site specific recombination with the third sequence, but is substantially incapable of homologous recombination with said target DNA sequence,
wherein the spatial order of said sequences in said DP vector is: said first homologous vector DNA sequence, said positive selection marker DNA sequence containing the third sequence, said second homologous vector DNA sequence and said fourth sequence;
wherein the vector is capable of modifying said target DNA sequence by homologous recombination of said first homologous vector DNA sequence with said first region of said target sequence and of said second homologous vector DNA sequence with said second region of said target sequence.

The double positive selection (“DP”) methods and vectors or targeting vectors of the invention are used to modify target DNA sequences in the genome of cells capable of homologous recombination.

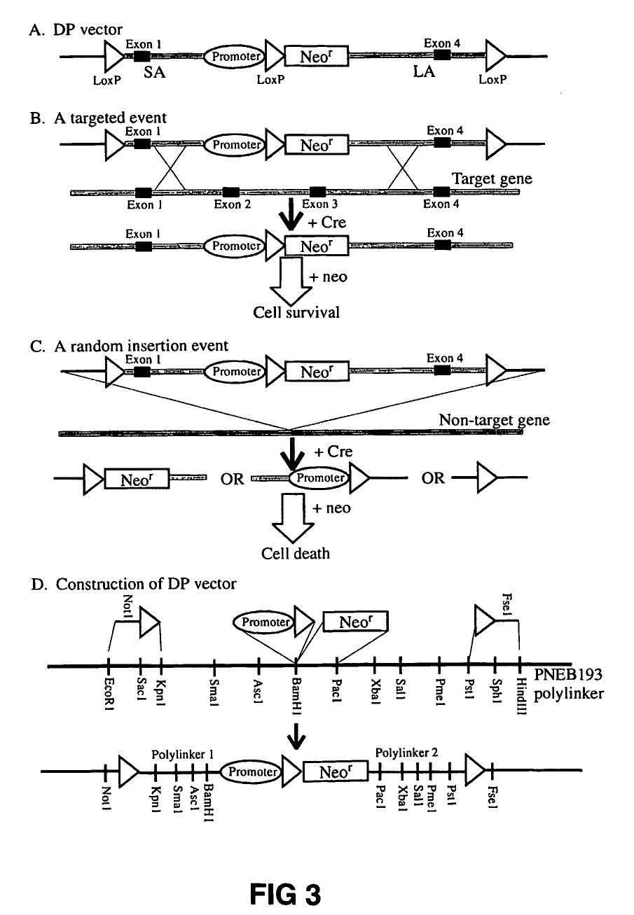

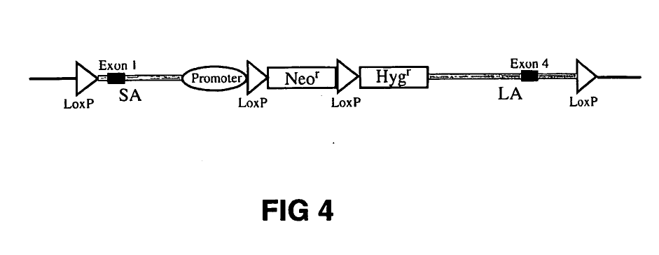

A schematic diagram of a DP vector of the invention is shown in FIG. 3 and FIG. 4. An alternate DP vector is shown in FIG. 4. The DP vector comprises at least four DNA sequences. The first and second DNA sequences each contain portions which are substantially homologous to corresponding homologous portions in first and second regions of the targeted DNA. Substantial homology is necessary between these portions in the DP vector and the target DNA to insure targeting of the DP vector to the appropriate region of the genome.

As used herein the term “homologous” or “substantially homologous” DNA sequence as used herein is a DNA sequence that is identical with or nearly identical with a reference DNA sequence. Indications that two sequences are homologous is that they will hybridize with each other even under the most stringent hybridization conditions; and preferably will not exhibit sequence polymorphisms (i.e. they will not have different sites for cleavage by restriction endonucleases).

The term “substantially homologous” as used herein refers to DNA that is at least about 97-99% identical with the reference DNA sequence, and preferably at least about 99.5-99.9% identical with the reference DNA sequence, and in certain uses 100% identical with the reference DNA sequence. Indications that two sequences are substantially homologous is that they will still hybridize with each other under the most stringent conditions and they will only rarely exhibit RFLPs or sequence polymorphisms (relative to the number that would be statistically expected for sequences of their particular length which share at least about 97-99% sequence identity).

Gene targeting represents a major advance in the ability to selectively manipulate animal cell genomes. Using this technique, a particular DNA sequence can be targeted and modified in a site-specific and precise manner. Different types of DNA sequences can be targeted for modification, including regulatory regions, coding regions and regions of DNA between genes. Examples of regulatory regions include: promoter regions, enhancer regions, terminator regions and introns. By modifying these regulatory regions, the timing and level of expression of a gene can be altered. Coding regions can be modified to alter, enhance or eliminate, for example, the specificity of an antigen or antibody, the activity of an enzyme, the composition of a food protein, the sensitivity of protein to inactivation, the secretion of a protein, or the routing of a protein within a cell. Introns and exons, as well as inter-genic regions, are suitable targets for modification. The technology when used in combination with recombinases also allows for chromosomal engineering (Ramirez-Solis R, Liu P, Bradley A (1995). Chromosome engineering in mice. Nature 378:7204.) whereby large inter-chromosomes or intra-chromosome rearrangements may be achieved.

Modifications of DNA sequences can be of several types, including insertions, deletions, substitutions, or any combination of the preceding. A specific example of a modification is the inactivation of a gene by site-specific integration of a nucleotide sequence that disrupts expression of the gene product. Using such a technique to “knock out” a gene by targeting will avoid problems associated with the use of antisense RNA to disrupt functional expression of a gene product. For example, one approach to disrupting a target gene using the present invention would be to insert a selectable marker into the targeting DNA such that homologous recombination between the targeting DNA and the target DNA will result in insertion of the selectable marker into the coding region of the target gene.

Also included in the DP vector is a DNA sequence which encodes a positive selection marker. Preferred positive selection markers as used herein the description, include, but is not limited to, drug resistance genes (eg neomycin-resistance, hygromycin-resistance etc), fluorescent or bioluminescent markers (eg green fluorescent protein (GFP), yellow fluorescent protein (YFP), etc) or any other marker that can be used to distinguish cells carrying the inserted DNA from cells lacking such DNA.

The DNA sequence encoding the positive selection marker is preferably positioned between the first and second DNA sequences. The preferred location of the marker gene in the targeting construct will depend on the aim of the gene targeting. For example, if the aim is to disrupt target gene expression, then the selectable marker can be cloned into targeting DNA corresponding to coding sequence in the target DNA. Alternatively, if the aim is to express an altered product from the target gene, such as a protein with an amino acid substitution, then the coding sequence can be modified to code for the substitution, and the selectable marker can be placed outside of the coding region, in a nearby intron for example.

If the selectable markers will depend on their own promoters for expression and the marker gene is derived from a very different organism than the organism being targeted (e.g. prokaryotic marker genes used in targeting mammalian cells), it is preferable to replace the original promoter with transcriptional machinery known to function in the recipient cells. A large number of transcriptional initiation regions are available for such purposes including, for example, metallothionein promoters, thymidine kinase promoters, beta-actin promoters, immunoglobulin promoters, SV40 promoters and cytomegalovirus promoters. A widely used example is the pSV2-neo plasmid which has the bacterial neomycin phosphotransferase gene under control of the SV40 early promoter and confers in mammalian cells resistance to G418 (an antibiotic related to neomycin).

A feature of the marker in the DP vector is that within the coding sequence of the positive marker there is a third sequence which supports high efficiency recombination in the presence of a site specific recombinase. A suitable sequence includes those of the loxP or inverted repeat sequences (FRTs) which are under the influence of recombinases such as Cre or FLP respectively. Other members of the Intergrase family of recombinases (Gln, Hin, resolvase) are also included in this description. Other enzymatically mediated or high efficiency recombination events can potentially mediate this system.

The loxP sequences are 34 base pair stretches of DNA which flank sequences which are to be deleted. It is incapable of recombination without the involvement of the Cre protein.

The FRT sequence is the target of FLP and will also flank DNA sequences which can be deleted in the presence of the recombinase FLP.

It is preferred that the third sequence which supports high-efficiency DNA recombination in the presence of a site-specific recombinase may be inserted without disrupting the expression of the positive selection marker gene when the promoter sequence is activated within a cell. This sequence is preferably inserted between the promoter and the coding region of the selection marker gene. However, alternate strategies may be devised, such as the strategy utilised in the Blue/White selection strategy whereby the P-galactosidase coding sequence is disrupted.

Positive markers are “functional” in transformed cells if the phenotype expressed by the DNA sequences encoding such selection markers is capable of conferring a positive selection characteristic for the cell expressing that DNA sequence. Thus, “positive selection” comprises introducing cells transfected with a DP vector with an appropriate agent which kills or otherwise selects against cells not containing an integrated positive selection marker.

Other positive selection markers used herein include DNA sequences encoding membrane bound polypeptides. Such polypeptides are well known to those skilled in the art and contain a secretory sequence, an extracellular domain, a transmembrane domain and an intracellular domain. When expressed as a positive selection marker, such polypeptides associate with the target cell membrane. Fluorescently labelled antibodies specific for the extracellular domain may then be used in a fluorescence activated cell sorter (FACS) to select for cells expressing the membrane bound polypeptide. FACS selection may occur before or after negative selection.

The fourth sequence in the DP vector supports high-efficiency DNA recombination in the presence of a site specific recombinase and directs site-specific recombination with the third sequence, but is substantially incapable of homologous recombination with the target DNA sequence. Preferably, where the third sequence is a loxP sequence, the fourth sequence is also a loxP sequence, thereby facilitating the removal of the sequence that they flank. Similarly, where the third sequence is a FRT sequence, the fourth sequence is also an FRT sequence, thereby facilitating the removal of the sequence that they flank. The respective recombinases are Cre and FLP. Other recombinases which can produce the same effect are within the scope of this application.

A key feature of this invention is that under the influence of a site-specific recombinase (eg Cre) recombination will occur between the loxP sites resulting in the loss in function of the positive-selection marker. Accordingly, the cells in which the homologous recombination is to occur must have their genome altered to express an inducible form of Cre (or alternate recombinase such as FLP).

In a further preferred embodiment the fourth DNA sequence contains another short region of DNA which may act as a PCR primer site or an alternative recombination site to that used within the positive selection marker. This may be added to either end of the first and second DNA sequence.

The positive selection marker, however, may be constructed so that it is independently expressed (eg. when contained in an intron of the target DNA) or constructed so that homologous recombination will place it under control of regulatory sequences in the target DNA sequence.

The positioning of the various DNA sequences within the DP vector, however, does not require that each of the DNA sequences be transcriptionally and translationally aligned on a single strand of the DP vector. Thus, for example, the first and second DNA sequences may have a 5′ to 3′ orientation consistent with the 5′ to 3′ orientation of regions 1 and 2 in the target DNA sequence. When so aligned, the DP vector is a “replacement DP vector”. Upon homologous recombination the replacement DP vector replaces the genomic DNA sequence between the homologous portions of the target DNA with the DNA sequences between the homologous portion of the first and second DNA sequences of the DP vector. Sequence replacement vectors are preferred in practicing the invention. Alternatively, the homologous portions of the first and second DNA sequence in the DP vector may be inverted relative to each other such that the homologous portion of DNA sequence 1 corresponds 5′ to 3′ with the homologous portion of region 1 of the target DNA sequence whereas the homologous portion of DNA sequence 2 in the DP vector has an orientation which is 3′ to 5′ for the homologous portion of the second region of the second region of the target DNA sequence. This inverted orientation provides for an “insertion DP vector”. When an insertion DP vector is homologously inserted into the target DNA sequence, the entire DP vector is inserted into the target DNA sequence without replacing the homologous portions in the target DNA. The modified target DNA so obtained necessarily contains the duplication of at least those homologous portions of the target DNA which are contained in the DP vector.

Similarly, the positive selection marker, third and fourth DNA sequences may be transcriptionally inverted relative to each other and to the transcriptional orientation of the target DNA sequence. This is only the case, however, when expression of the positive selection marker in the third DNA sequence is independently controlled by appropriate regulatory sequences. When, for example a promoterless positive selection marker is used as a third sequence such that its expression is to be placed under control of an endogenous regulatory region, such a vector requires that the positive selection marker be positioned so that it is in proper alignment (5′ to 3′ and proper reading frame) with the transcriptional orientation and sequence of the endogenous regulatory region.

DP selection requires that the fourth DNA sequence be substantially incapable of homologous recombination with the target DNA sequence.

In yet another aspect of the present invention there is another selection marker included on the vector. Preferably the additional marker is flanked by site specific recombination sequences which are under the influence of recombinases as described above for loxP and FRTs. The selection markers contained in such a DP vector may either be the same or different selection markers. When they are different such that they require the use of two different agents to select against cells containing such markers, such selection may be carried out sequentially or simultaneously with appropriate agents for the selection marker. The positioning of two selection markers at the 5′ and 3′ end of a DP vector further enhances selection against target cells which have randomly integrated the DP vector. This is because random integration sometimes results in the rearrangement of the DP vector resulting in excision of all or part of the selection marker prior to random integration. When this occurs, cells randomly integrating the DP vector cannot be selected against. However, the presence of a second selection marker on the DP vector substantially enhances the likelihood that random integration will result in the insertion of at least one of the two selection markers.

Preferably, the invention includes an alternate selection marker that is flanked by loxP sites that can be used to exclude cells in which the Cre is non-functional during the DP selection process. This marker may be any marker as described above, preferably a herpes simplex thymidine kinase or a fluorescent protein etc. The inducible Cre would be activated after a period of time and would allow for homologous recombination to occur in most cells. This would vary between cell types. The selection for the selection marker would be initiated some time after induction of Cre, and would act to exclude cells in which Cre was not. functioning efficiently.

In a further preferred embodiment of this aspect, there is provided a DP vector with at least two selectable markers wherein one of the markers is a promoter-less marker and the other marker is under the influence of a promoter. A suitable promoter-less marker is a hygromycin resistance marker (Hyg^{r}).

In a system that relies on the expression of the Cre recombinase in 100% or nearly 100% of cells so that all cells with a random integration event become sensitive to a first selection marker and hence be killed in a second round of the selection, there can be no cells that do not have 100% efficiency. Otherwise the selection procedure produces background carrying random integrations. Accordingly, to reduce the incidence of the random integration, a form of the DP vector is provided in this invention, said vector including at least two positive selectable markers (FIG. 4).

This vector is the same as the DP vector described above except that another LoxP site and a promoter-less selectable marker resistance gene will be present after the first selection marker gene. The promoter-less marker gene will not be expressed because the first marker gene serves as a “stuffer sequence” between the promoter and the promoter-less gene. This vector will be used to transfect cells selecting for the first marker resistance. After a targeted event, expression of the Cre recombinase using the adenovirus-Cre will delete the first section marker gene, allowing the expression of the promoter-less gene, conferring the cells or a different resistance. In a random integration event with the two outside LoxP sites present, expression of Cre recombinase will delete the promoter or the promoter-less gene (or both). Therefore, using promoter-less the second selection, the survivors should only be those cells resulting from a targeted event. Inefficient expression of the Cre recombinase is not a problem here because the promoter-less gene will not be expressed without LoxP recombination. This modification represents a superior gene targeting vector that will produce a much lower level of non-targeted cells.

The substantial non-homology between the fourth DNA sequence of the DP vector and the target DNA creates a discontinuity in sequence homology at or near the juncture of the fourth DNA sequence. Thus, when the vector is integrated into the genome by way of the homologous recombination mechanism of the cell, the fourth DNA sequence is not transferred into the target DNA. It is the non-integration of this fourth DNA sequence during homologous recombination and the activation of a recombinase which forms the basis of the DP method of the invention.

As used herein, a “modifying DNA sequence” is a DNA sequence contained in the first, second and/or positive selection marker DNA sequence which encodes the substitution, insertion and/or deletion of one or more nucleotides in the target DNA sequence after homologous insertion of the DP vector into the targeted region of the genome. When the DP vector contains only the insertion of the DNA sequence encoding the positive selection marker, the DNA sequence is sometimes referred to as a “first modifying DNA sequence”. When in addition to the DNA sequence which encodes the selection marker, the DP vector also encodes the further substitution, insertion and/or deletion of one or more nucleotides, that portion encoding such further modification is sometimes referred to as a “second modifying DNA sequence”. The second modifying DNA sequence may comprise the entire first and/or second DNA sequence or in some instances may comprise less than the entire first and/or second DNA sequence. The latter case typically arises when, for example, a heterologous gene is incorporated into a DP vector which is designed to place that heterologous gene under the regulatory control of endogenous regulatory sequences. In such a case, the homologous portion of, for example, the first DNA sequence may comprise all or part of the targeted endogenous regulatory sequence and the modifying DNA sequence comprises that portion of the first DNA sequence (and in some cases a part of the second DNA sequence as well) which encodes the heterologous DNA sequence. An appropriate homologous portion in the second DNA sequence will be included to complete the targeting of the DP vector. On the other hand, the entire first and/or second DNA sequence may comprise a second modifying DNA sequence when, for example, either or both of these DNA sequences encode for the correction of a genetic defect in the targeted DNA sequence.

In a further preferred embodiment, the present invention provides a DP vector comprising a targeting construct prepared by a transposon mediated method, said method comprising the steps of:
obtaining a copy of the target DNA sequence in vitro;
inserting a DNA sequence comprising a transposon sequence and a DNA recombination sequence at one site in the copy of the target DNA sequence;
inserting another DNA sequence comprising a transposon sequence and a DNA recombination sequence at another site in the copy of the target DNA sequence;
inducing a recombination event between said recombination sequences to delete a portion of the copy of the target DNA sequence.

Following the recombination step, it is preferred that the targeting construct is recovered and isolated for insertion into the DP vector. Recovering may be achieved by any method that isolates the construct from its cloning vector. Restriction digests are desired to cut the construct in a manner which facilitates insertion into the DP vector.

In an even further preferred embodiment, the targeting construct comprises:
a first homologous vector DNA sequence capable of homologous recombination with a first region of said target DNA sequence;
a positive selection marker DNA sequence capable of conferring a positive selection characteristic in said cells;
a third sequence that supports high-efficiency DNA recombination in the presence of a site specific recombinase and which is contained within the positive selection marker;
a second homologous vector DNA sequence capable of homologous recombination with a second region of said target DNA sequence; and
a fourth sequence which directs site specific recombination with the third sequence, but is substantially incapable of homologous recombination with said target DNA sequence;
wherein the vector is capable of modifying said target DNA sequence by homologous recombination of said first homologous vector DNA sequence with said first region of said target sequence and of said second homologous vector DNA sequence with said second region of said target sequence.

As used herein, “modified target DNA sequence” refers to a DNA sequence in the genome of a targeted cell which has been modified by a DP vector. Modified DNA sequences contain the substitution, insertion and/or deletion of one or more nucleotides in a first transformed target cell as compared to the cells from which such transformed target cells are derived. In some cases, modified target DNA sequences are referred to as “first” and/or “second modified target DNA sequences”. These correspond to the DNA sequence found in the transformed target cell when a DP vector containing a first or second modifying sequence is homologously integrated into the target DNA sequence.

“Transformed target cells” sometimes referred to as “first transformed target cells” refers to those target cells wherein the DP vector has been homologously integrated into the target cell genome. A “transformed cell” on the other hand refers to a cell wherein the DP has non-homologously inserted into the genome randomly. “Transformed target cells” generally contain a positive selection marker within the modified target DNA sequence. When the object of the genomic modification is to disrupt the expression of a particular gene, the positive selection marker is generally contained within an exon which effectively disrupts transcription and/or translation of the targeted endogenous gene. When, however, the object of the genomic modification is to insert an exogenous gene or correct an endogenous gene defect, the modified target DNA sequence in the first transformed target cell will in addition contain exogenous DNA sequences or endogenous DNA sequences corresponding to those found in the normal, i.e., nondefective, endogenous gene.

“Second transformed target cells” refers to first transformed target cells whose genome has been subsequently modified in a predetermined way. For example, the positive selection marker contained in the genome of a first transformed target cell can be excised by homologous recombination to produce a second transformed target cell. The details of such a predetermined genomic manipulation will be described in more detail hereinafter.

As used herein, “heterologous DNA” refers to a DNA sequence which is different from that sequence comprising the target DNA sequence. Heterologous DNA differs from target DNA by the substitution, insertion and/or deletion of one or more nucleotides. Thus, an endogenous gene sequence may be incorporated into a DP vector to target its insertion into a different regulatory region of the genome of the same organism. The modified DNA sequence so obtained is a heterologous DNA sequence. Heterologous DNA sequences also include endogenous sequences which have been modified to correct or introduce gene defects or to change the amino acid sequence encoded by the endogenous gene. Further, heterologous DNA sequences include exogenous DNA sequences which are not related to endogenous sequences, e.g. sequences derived from a different species. Such “exogenous DNA sequences” include those which encode exogenous polypeptides or exogenous regulatory sequences. For example, exogenous DNA sequences which can be introduced into murine or bovine ES cells for tissue specific expression (e.g. in mammary secretory cells) include human blood factors such as t-PA, Factor VIII, serum albumin and the like. DNA sequences encoding positive selection markers are further examples of heterologous DNA sequences.

In yet another aspect of the present invention there is provided a method for enriching for a transformed cell containing a modification in a target DNA sequence in the genome of said cell comprising:
(a) transfecting cells capable of mediating homologous recombination with a DP selection vector said vector comprising
   a first homologous vector DNA sequence capable of homologous recombination with a first region of said target DNA sequence;
a positive selection marker DNA sequence capable of conferring a positive selection characteristic in said cells;
   a third sequence that supports DNA recombination in the presence of a site specific recombinase and which is contained within the positive selection marker;
   a second homologous vector DNA sequence capable of homologous recombination with a second region of said target DNA sequence; and
   a fourth sequence which directs site specific recombination with the third sequence, but is substantially incapable of homologous recombination with said target DNA sequence,
   wherein the spatial order of said sequences in said DP vector is: said first homologous vector DNA sequence, said positive selection marker DNA sequence containing the third sequence, said second homologous vector DNA sequence and said fourth sequence; and
   wherein the vector is capable of modifying said target DNA sequence by homologous recombination of said first homologous vector DNA sequence with said first region of said target sequence and of said second homologous vector DNA sequence with said second region of said target sequence.
(b) selecting for transformed cells in which said DP selection vector has integrated into said target DNA sequence by homologous recombination by sequentially or simultaneously selecting for transformed cells containing the positive selection marker in the presence of the recombinase; and
(c) analysing the DNA of transformed cells surviving the selecting step to identify a cell containing the modification.

The selection of desired homologous recombination events is based on distinguishing between targeted and random events. In a targeted event recombination occurs with the target DNA sequence and the first and second DNA sequences, resulting in the exclusion of the loxP sites or FRT sites at either end of the vector. Therefore, in the presence of Cre (or alternate recombinase such as FLP) recombination events will not take place and the positive selection marker will remain intact and functional. Hence ongoing maintenance of the positive-selection of cells expressing the marker DNA will result in survival of the cells.

Alternately, in a random event, one or both ends of the vector will (generally) remain intact leaving two or three functional loxP sites or FRTs being incorporated into the cell's genome. Activation of the inducible Cre (or alternate recombinase such as FLP) within the cell will result in the positive selection marker becoming non-functional. Hence, the ongoing maintenance of the cells under positive-selection conditions will result in the death or exclusion of such cells from the selection process.

In some circumstances the recombination event will be inefficient resulting in a relatively inefficient rate of exclusion on non-homologous recombination events. The primer sequence will function in these cases to enable detection of relatively rare events that would be detectable by PCR reactions. Hence at the end of the DP selection process a cell or cells would be continued to be maintained under Cre allowing for ongoing but infrequent rates of recombination. to occur—which can be detected by a PCR reaction using primers specific to the Primer Sequence and the First or Second DNA sequence.

The DP vector is used in the DP method to select for transformed target cells containing the positive selection marker. Such positive selection procedures substantially enrich for those transformed target cells wherein homologous recombination has occurred. As used herein, “substantial enrichment” refers to at least a two-fold enrichment of transformed target cells as compared to the ratio of homologous transformants versus non-homologous transformants, preferably a 10-fold enrichment, more preferably a 1000-fold enrichment, most preferably a 10,000-fold enrichment, i.e., the ratio of transformed target cells to transformed cells. In some instances, the frequency of homologous recombination versus random integration is of the order of 1 in 1000 and in some cases as low as 1 in 10,000 transformed cells. The substantial enrichment obtained by the DP vectors and methods of the invention often result in cell populations wherein about 1 %, and more preferably about 20%, and most preferably about 95% of the resultant cell population contains transformed target cells wherein the DP vector has been homologously integrated. Such substantially enriched transformed target cell populations may thereafter be used for subsequent genetic manipulation, for cell culture experiments or for the production of transgenic organisms such as transgenic animals or plants.

In a preferred embodiment, the DP selection vector comprises a targeting construct prepared by the transposon-mediated method as herein described.

In yet another aspect of the present invention, there is provided a transformed cell prepared by the methods described herein.

The cells may be any prokaryotic or eukaryotic cells which are capable of receiving the DP vector and being modified with the target DNA.

In an even further aspect of the present invention there is provided a method of inducing a modification in genome of a cell, said method comprising:
transfecting cells capable of mediating homologous recombination with a DP selection vector said vector comprising
a first homologous vector DNA sequence capable of homologous recombination with a first region of said target DNA sequence;
a positive selection marker DNA sequence capable of conferring a positive selection characteristic in said cells;
a third sequence that supports DNA recombination in the presence of a site specific recombinase and which is contained within the positive selection marker;
a second homologous vector DNA sequence capable of homologous recombination with a second region of said target DNA sequence; and
a fourth sequence which directs site specific recombination with the third sequence, but is substantially incapable of homologous recombination with said target DNA sequence,
wherein the spatial order of said sequences in said DP vector is: said first homologous vector DNA sequence, said positive selection marker DNA sequence containing the third sequence, said second homologous vector DNA sequence and said fourth sequence;
wherein the vector is capable of modifying said target DNA sequence by homologous recombination of said first homologous vector DNA sequence with said first region of said target sequence and of said second homologous vector DNA sequence with said second region of said target sequence.

In a preferred embodiment, the DP selection vector comprises a targeting construct prepared by the transposon mediated method as described herein.

The modification may include the deletion of a gene or replacement of a gene sequence. The modification may be a predetermined modification of target DNA sequence.

In many cases it is desirable to disrupt genes by positioning the positive selection marker in an exon of a gene to be disrupted or modified. For example, specific proto-oncogenes can be mutated by this method to produce transgenic animals. Such transgenic animals containing selectively inactivated proto-oncogenes are useful in dissecting the genetic contribution of such a gene to oncogenesis and in some cases normal development.

Another potential use for gene inactivation is disruption of proteinaceous receptors on cell surfaces. For example, cell lines or organisms wherein the expression of a putative viral receptor has been disrupted using an appropriate DP vector can be assayed with virus to confirm that the receptor is, in fact, involved in viral infection. Further, appropriate DP vectors may be used to produce transgenic animal models for specific genetic defects. For example, many gene defects have been characterized by the failure of specific genes to express functional gene product, e.g. a and β thalassemia, hemophilia, Gaucher's disease and defects affecting the production of α-1-antitrypsin, ADA, PNP, phenylketonuria, familial hypercholesterolemia and retinoblastoma. Transgenic animals containing disruption of one or both alleles associated with such disease states or modification to encode the specific gene defect can be used as models for therapy. For those animals which are viable at birth, experimental therapy can be applied. When, however, the gene defect affects survival, an appropriate generation (e.g. F0, F1) of transgenic animal may be used to study in vivo techniques for gene therapy.

A modification of the foregoing means to disrupt gene X by way of homologous integration involves the use of a positive selection marker which is deficient in one or more regulatory sequences necessary for expression. The DP vector is constructed so that part but not all of the regulatory sequences for gene X are contained in the DP vector 5′ from the structural gene segment encoding the positive selection marker, e.g., homologous sequences encoding part of the promoter of the X gene. As a consequence of this construction, the positive selection marker is not functional in the target cell until such time as it is homologously integrated into the promoter region of gene X. When so integrated, gene X is disrupted and such cells may be selected by way of the positive selection marker expressed under the control of the target gene promoter. The only limitation in using such an approach is the requirement that the targeted gene be actively expressed in the cell type used. Otherwise, the positive selection marker will not be expressed to confer a positive selection characteristic on the cell.

In many instances, the disruption of an endogenous gene is undesirable, e.g., for some gene therapy applications. In such situations, the positive selection marker of the DP vector may be positioned within an untranslated sequence, e.g. an intron of the target DNA or 5′ or 3′ untranslated regions. The positive selection marker is positioned between the first and second sequences. The fourth DNA sequence is positioned outside of the region of homology. When the DP vector is integrated into the target DNA by way of homologous recombination the positive selection marker is located in the intron of the targeted gene. The selection marker sequence is constructed such that it is capable of being expressed and translated independently of the targeted gene. Thus, it contains an independent functional promoter, translation initiation sequence, translation termination sequence, and in some cases a polyadenylation sequence and/or one or more enhancer sequences, each functional in the cell type transfected with the DP vector. In this manner, cells incorporating the DP vector by way of homologous recombination can be selected by way of the positive selection marker without disruption of the endogenous gene. Of course, the same regulatory sequences can be used to control the expression of the positive selection marker when it is positioned within an exon. Of course, other regulatory sequences may be used which are known to those skilled in the art. In each case, the regulatory sequences will be properly aligned and, if necessary, placed in proper reading frame with the particular DNA sequence to be expressed. Regulatory sequence, e.g. enhancers and promoters from different sources may be combined to provide modulated gene expression.

There are, of course, numerous other examples of modifications of target DNA sequences in the genome of the cell which can be obtained by the DP vectors and methods of the invention. For example, endogenous regulatory sequences controlling the expression of proto-oncogenes can be replaced with regulatory sequences such as promoters and/or enhancers which actively express a particular gene in a specific cell type in an organism, i.e., tissue-specific regulatory sequences. In this manner, the expression of a proto-oncogene in a particular cell type, for example in a transgenic animal, can be controlled to determine the effect of oncogene expression in a cell type which does not normally express the proto-oncogene. Alternatively, known viral oncogenes can be inserted into specific sites of the target genome to bring about tissue-specific expression of the viral oncogene.

As indicated, the DP selection methods and vectors of the invention are used to modify target DNA sequences in the genome of target cells capable of homologous recombination. Accordingly, the invention may be practiced with any cell type which is capable of homologous recombination. Examples of such target cells include cells derived from vertebrates including mammals such as humans, bovine species, ovine species, murine species, simian species, and other eukaryotic organisms such as filamentous fungi, and higher multicellular organisms such as plants. The invention may also be practiced with lower organisms such as gram positive and gram negative bacteria capable of homologous recombination. However, such lower organisms are not preferred because they generally do not demonstrate significant non-homologous recombination, i.e., random integration. Accordingly, there is little or no need to select against non-homologous transformants.

In those cases where the ultimate goal is the production of a non-human transgenic animal, embryonic stem cells (ES cells) and neural stem cells are preferred target cells. ES cells may be obtained from pre-implantation embryos cultured in vitro. DP vectors can be efficiently introduced into the ES cells by electroporation or microinjection or other transformation methods, preferably electroporation. Such transformed ES cells can thereafter be combined with blastocysts from a non-human animal. The ES cells thereafter colonize the embryo and can contribute to the germ line of the resulting chimeric animal. In the present invention, DP vectors may be targeted to a specific portion of the ES cell genome and thereafter used to generate chimeric transgenic animals by standard techniques.

When the ultimate goal is gene therapy to correct a genetic defect in an organism such as a human being, the cell type will be determined by the aetiology of the particular disease and how it is manifested. For example, hemopoietic stem cells are a preferred cells for correcting genetic defects in cell types which differentiate from such stem cells, e.g. erythrocytes and leukocytes. Thus, genetic defects in globin chain synthesis in erythrocytes such as sickle cell anaemia, β-thalassemia and the like may be corrected by using the DP vectors and methods of the invention with hematopoietic stem cells isolated from an affected patient. For example, if the target DNA is the sickle-cell β-globin gene contained in a hematopoietic stem cell and the DP vector is targeted for this gene, transformed hematopoietic stem cells can be obtained wherein a normal β-globin will be expressed upon differentiation. After correction of the defect, the hematopoietic stem cells may be returned to the bone marrow or systemic circulation of the patient to form a subpopulation of erythrocytes containing normal haemoglobin. Alternatively, hematopoietic stem cells may be destroyed in the patient by way of irradiation and/or chemotherapy prior to reintroduction of the modified hematopoietic stem cell thereby rectifying the defect.

Other types of stem cells may be used to correct the specific gene defects associated with cells derived from such stem cells. Such other stem cells include epithelial, liver, lung, muscle, endothelial, mesenchymal, neural and bone stem cells.

Alternatively, certain disease states can be treated by modifying the genome of cells in a way that does not correct a genetic defect per se but provides for the supplementation of the gene product of a defective gene. For example, endothelial cells are preferred as targets for human gene therapy to treat disorders affecting factors normally present in the systemic circulation. In model studies using both dogs and pigs endothelial cells have been shown to form primary cultures, to be transformable with DNA in culture, and to be capable of expressing a transgene upon re-implantation in arterial grafts into the host organism. Since endothelial cells form an integral part of the graft, such transformed cells can be used to produce proteins to be secreted into the circulatory system and thus serve as therapeutic agents in the treatment of genetic disorders affecting circulating factors. Examples of such diseases include insulin-deficient diabetes, a-1-antitrypsin deficiency, and haemophilia. Epithelial cells provide a particular advantage in the treatment of factor VIII-deficient haemophilia. These cells naturally produce von Willebrand factor (vWF) and it has been shown that production of active factor VIII is dependant upon the autonomous synthesis of vWF.

Other diseases of the immune and/or the circulatory system are candidates for human gene therapy. The target tissue, bone marrow, is readily accessible by current technology, and advances are being made in culturing stem cells in vitro. The immune deficiency diseases caused by mutations in the enzymes adenosine deaminase (ADA) and purine nucleotide phosphorylase (PNP), are of particular interest. Not only have the genes been cloned, but cells corrected by DP gene therapy are likely to have a selective advantage over their mutant counterparts. Thus, ablation of the bone marrow in recipient patients may not be necessary.

The DP selection approach is applicable to genetic disorders with the following characteristics: first, the DNA sequence and preferably the cloned normal gene must be available; second, the appropriate, tissue relevant, stem cell or other appropriate cell must be available.

As indicated, genetic defects may be corrected in specific cell lines by positioning the positive selection marker in an untranslated region such as an intron near the site of the genetic defect together with flanking segments to correct the defect. In this approach, the positive selection marker is under its own regulatory control and is capable of expressing itself without substantially interfering with the expression of the targeted gene. In the case of human gene therapy, it may be desirable to introduce only those DNA sequences which are necessary to correct the particular genetic defect. In this regard, it is desirable, although not necessary, to remove the residual positive selection marker which remains after correction of the genetic defect by homologous recombination.

The DP vectors and methods of the invention are also applicable to the manipulation of plant cells and ultimately the genome of the entire plant. A wide variety of transgenic plants have been reported, including herbaceous dicots, woody dicots and monocots. A number of different gene transfer techniques have been developed for producing such transgenic plants and transformed plant cells. One technique used Agrobacterium tumefaciens as a gene transfer system. Rogers, et al. (1986), Methods Enzymol., 118, 627-640. A closely related transformation utilizes the bacterium Agrobacterium rhizogenes. In each of these systems a Ti or Ri plant transformation vector can be constructed containing border regions which define the DNA sequence to be inserted into the plant genome. These systems previously have been used to randomly integrate exogenous DNA to plant genomes. In the present invention, an appropriate DP vector may be inserted into the plant transformation vector between the border sequences defining the DNA sequences transferred into the plant cell by the Agrobacterium transformation vector.

Preferably, the DP vector of the invention is directly transferred to plant protoplasts by way of methods analogous to that previously used to introduce transgenes into protoplasts. Alternatively, the DP vector is contained within a liposome which may be fused to a plant protoplast or is directly inserted to plant protoplast by way of intranuclear microinjection. Microinjection is the preferred method for transfecting protoplasts. DP vectors may also be microinjected into meristematic inflorenscences. Finally, tissue explants can be transfected by way of a high velocity microprojectile coated with the DP vector analogous to the methods used for insertion of transgenes. Such transformed explants can be used to regenerate for example various serial crops.

Once the DP vector has been inserted into the plant cell by any of the foregoing methods, homologous recombination targets the DP vector to the appropriate site in the plant genome. Depending upon the methodology used to transfect, positive-negative selection is performed on tissue cultures of the transformed protoplast or plant cell. In some instances, cells amenable to tissue culture may be excised from a transformed plant either from the F₀ or a subsequent generation.

The DP vectors and method of the invention are used to precisely modify the plant genome in a predetermined way. Thus, for example, herbicide, insect and disease resistance may be predictably engineered into a specific plant species to provide, for example, tissue specific resistance, e.g., insect resistance in leaf and bark. Alternatively, the expression levels of various components within a plant may be modified by substituting appropriate regulatory elements to change the fatty acid and/or oil content in seed, the starch content within the plant and the elimination of components contributing to undesirable flavours in food. Alternatively, heterologous genes may be introduced into plants under the predetermined regulatory control in the plant to produce various hydrocarbons including waxes and hydrocarbons used in the production of rubber.

The amino acid composition of various storage proteins in wheat and corn, for example, which are known to be deficient in lysine and tryptophan may also be modified. DP vectors can be readily designed to alter specific codons within such storage proteins to encode lysine and/or tryptophan thereby increasing the nutritional value of such crops.

It is also possible to modify the levels of expression of various positive and negative regulatory elements controlling the expression of particular proteins in various cells and organisms. Thus, the expression level of negative regulatory elements may be decreased by use of an appropriate promoter to enhance the expression of a particular protein or proteins under control of such a negative regulatory element. Alternatively, the expression level of a positive regulatory protein may be increased to enhance expression of the regulated protein or decreased to reduce the amount of regulated protein in the cell or organism.

The basic elements of the DP vectors of the invention have already been described. The selection of each of the DNA sequences comprising the DP vector, however, will depend upon the cell type used, the target DNA sequence to be modified and the type of modification which is desired.

Preferably, the DP vector is a linear double stranded DNA sequence. However, circular closed DP vectors may also be used. Linear vectors are preferred since they enhance the frequency of homologous integration into the target DNA sequence. (Thomas, et al. (1986), Cell, 44, 49).

In general, the DP vector has a total length of between 2.5 kb (2500 base pairs) and 1000 kb. The lower size limit is set by two criteria. The first of these is the minimum necessary length of homology between the first and second sequences of the DP vector and the target locus. This minimum is approximately 500 bp (DNA sequence 1 plus DNA sequence 2). The second criterion is the need for functional genes in the third. Finally a small additional length is required for targeted recombination sites (eg LoxP sites are 34 bp in length). For practical reasons, this lower limit is approximately 1000 bp for each sequence. This is because the smallest DNA sequences encoding known positive and negative selection markers are about 1.0-1.5 kb in length.

The upper limit to the length of the DP vector is determined by the state of the technology used to manipulate DNA fragments. If these fragments are propagated as bacterial plasmids, a practical upper length limit is about 25 kb; if propagated as cosmids, the limit is about 50 kb, if propagated as YACs (yeast artificial chromosomes) the limit approaches 2000 kb (eg the CEPH B YACs can be this size).

Within the first and second DNA sequences of the DP vector are portions of DNA sequence which are substantially homologous with sequence portions contained within the first and second regions of the target DNA sequence. The degree of homology between the vector and target sequences influences the frequency of homologous recombination between the two sequences. One hundred percent sequence homology is most preferred, however, lower sequence homology can be used to practice the invention. Thus, sequence homology as low as about 80% can be used. A practical lower limit to sequence homology can be defined functionally as that amount of homology which if further reduced does not mediate homologous integration of the DP vector into the genome. Although as few as 25 bp of 100% homology are required for homologous recombination in mammalian cells (Ayares, et al. (1986), Genetics, 83, 5199-5203), longer regions are preferred, e.g., 500 bp, more preferably, 5000 bp, and most preferably, 25000 bp for each homologous portion. These numbers define the limits of the individual lengths of the first and second sequences. Preferably, the homologous portions of the DP vector will be 100% homologous to the target DNA sequence, as increasing the amount of non-homology will result in a corresponding decrease in the frequency of gene targeting. If non-homology does exist between the homologous portion of the DP vector and the appropriate region of the target DNA, it is preferred that the non-homology not be spread throughout the homologous portion but rather in discrete areas of the homologous portion. It is also preferred that the homologous portion of the DP vector adjacent to the fourth sequence be 100% homologous to the corresponding region in the target DNA. This is to ensure maximum discontinuity between homologous and non-homologous sequences in the DP vector.

Increased frequencies of homologous recombination have been observed when the absolute amount of DNA sequence in the combined homologous portions of the first and second DNA sequence are increased.

As previously indicated, the fourth DNA should have sufficient non-homology to the target DNA sequence to prevent homologous recombination between the fourth DNA sequence and the target DNA. This is generally not a problem since it is unlikely that the negative selection marker chosen will have any substantial homology to the target DNA sequence. In any event, the sequence homology between the fourth DNA sequence and the target DNA sequence should be less than about 50%, most preferably less than about 30%.

A preliminary assay for sufficient sequence non-homology between the fourth DNA sequence and the target DNA sequence utilizes standard hybridization techniques. For example, the particular negative selection marker may be appropriately labelled with a radioisotope or other detectable marker and used as a probe in a Southern blot analysis of the genomic DNA of the target cell. If little or no signal is detected under intermediate stringency conditions such as 3×SSC when hybridized at about 55° C., that fourth sequence should be functional in a DP vector designed for homologous recombination in that cell type. However, even if a signal is detected, it is not necessarily indicative that particular fourth sequence cannot be used in a DP vector targeted for that genome. This is because the sequence may be hybridizing with a region of the genome which is not in proximity with the target DNA sequence.

It is also possible that high stringency hybridization can be used to ascertain whether genes from one species can be targeted into related genes in a different species. For example, preliminary gene therapy experiments may require that human genomic sequences replace the corresponding related genomic sequence in mouse cells. High stringency hybridization conditions such as 0.1×SSC at about 68 degree C. can be used to correlate hybridization signal under such conditions with the ability of such sequences to act as homologous portions in the first and second DNA sequence of the DP vector. Such experiments can be routinely performed with various genomic sequences having known differences in homology. The measure of hybridization may therefore correlate with the ability of such sequences to bring about acceptable frequencies of recombination.

In another aspect there is provided a method of producing a transgenic plant or animal having a genome comprising a modification of a target DNA sequence, said method comprising:
transforming a population of embryonic stem cells with a DP vector;
identifying a cell having said genome by selecting for cells containing said DP vector and analysing DNA from cells surviving selection for the presence of the modification;
inserting the cell into an embryo;
propagating a plant or animal from the embryo;
wherein the DP vector comprises:
a first homologous vector DNA sequence capable of homologous recombination with a first region of said target DNA sequence;
a positive selection marker DNA sequence capable of conferring a positive selection characteristic in said cells;
a third sequence that supports DNA recombination in the presence of a site specific recombinase and which is contained within the positive selection marker;
a second homologous vector DNA sequence capable of homologous recombination with a second region of said target DNA sequence; and
a fourth sequence which directs site specific recombination with the third sequence, but is substantially incapable of homologous recombination with said target DNA sequence,
wherein the spatial order of said sequences in said DP vector is: said first homologous vector DNA sequence, said positive selection marker DNA sequence containing the third sequence, said second homologous vector DNA sequence and said fourth sequence;
wherein the vector is capable of modifying said target DNA sequence by homologous recombination of said first homologous vector DNA sequence with said first region of said target sequence and of said second homologous vector DNA sequence with said second region of said target sequence.

Preferably, the DP vector comprises a targeting construct prepared by the transposon mediated methods described herein.

The embryonic stem cells may be derived from any animal or plant and may be isolated and prepared by any methods available to the skilled addressee

The DP vectors are preferably as described above.

In an even further aspect of the present invention, there is provided a transgenic animal or plant prepared by the methods described herein.

The present invention will now be more fully described with reference to the accompanying examples and figures. It should be understood, however, that the description following is illustrative only and should not be taken in any way as a restriction of the generality of the invention described.

### EXAMPLES

### Example 1

### Development of a Transposon-Mediated Procedure to Generate Deletions

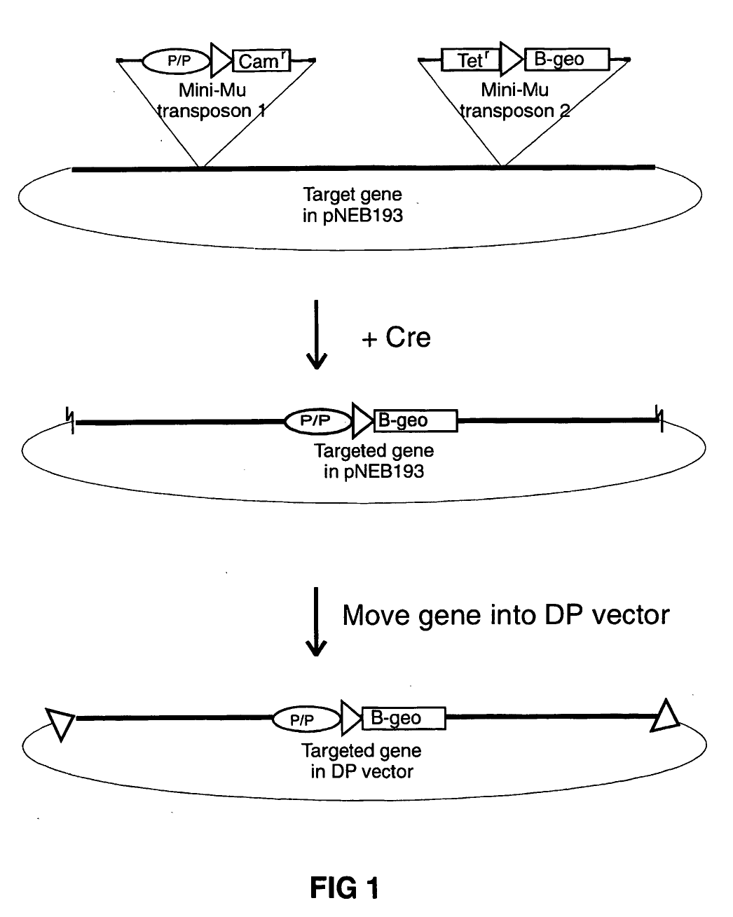

For the generation of deletions in cloned genes, two mini-Mu transposons may be constructed using the procedure previously developed (Haapa et al., 1999 Nucleic Acid Res. 27:2777-2784). The construction of suitable transposons and their use in the generation of deletions are shown in FIG. 1. The β-geo marker is used here only as an example and other markers are equally suitable. The use of Mini-Mu transposons have also been illustrated, however other transposons may be equally useful.

Mini-Mu transposon 1 may be constructed as follows. The prokaryotic/eukaryotic double promoter (P/P) may be amplified from a Clontech vector such as pEGFP-N1, incorporating the transposon end at the 5′-end and a LoxP sequence at the 3′-end. The chloramphenicol resistance gene (Cam^{r}) may be amplified from a vector such as pACYC184, incorporating the transposon end at the 3′-end. The former PCR product may be ligated 5′ to the latter and the composite construct cloned into the polylinker of pUC19. In this transposon, the bacterial promoter will drive the expression of the Cam^{r} gene.

Mini-Mu transposon 2 may be constructed as follows. The tetracycline resistance gene (Tet^{r}) may be amplified from a plasmid such as pBR322, incorporating the transposon end at the 5′-end and a LoxP sequence at the 3′-end. The promoter-less β-geo gene may be amplified from a vector such as pβAclβgeo incorporating the transposon end at the 3′-end. The former PCR product may be ligated 5′ to the latter and the composite construct cloned into the polylinker of pUC19. In this transposon, the Tet^{r} gene may be expressed in E. coli and the β-geo gene will not be expressed at its present form.

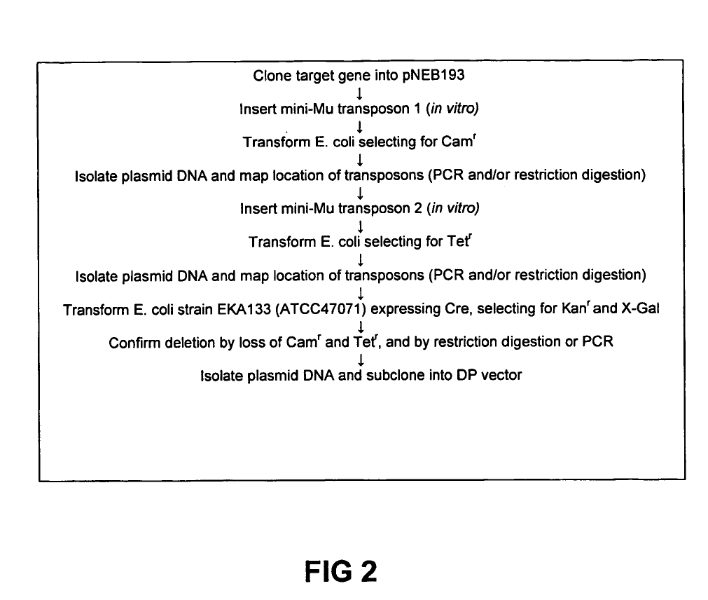

A general schematic of application of this technology is presented in FIG. 2. The target gene may be cloned into pNEB193 and the two transposons inserted at the desired positions with the right orientation. Mini-Mu transposon 1 may be inserted into the cloned gene by in vitro transposition, the transposition mixture transformed into E. coli cells selecting for Cam^{r}. The transposon insertions may be mapped physically and the one at the first desired deletion point selected (this need to be in the orientation shown in the FIG. 1). Mini-Mu transposon 2 may then be inserted into the clone, selecting for Tet^{r} in E. coli. The transposon insertions may be mapped and the one at the second desired deletion point selected (this needs to be in the orientation shown in the FIG. 1).

The selected clone may be transformed into the E. coli strain EAK133 expressing the cre recombinase. This will generate a deletion between the two LoxP sites, eliminating the Cam^{r} and Tet^{r} genes as well as the part of the target animal gene between the two transposons. These cells can be selected by Kan^{r} and the blue colour in the presence of X-Gal because the promoter on mini-Mu transposon 1 will now drive the expression of the β-geo gene. The deletion events can be confirmed by the loss of Cam^{r} and Tet^{r}, and by restriction digestion or PCR.

The gene construct may be excised from the vector and cloned between the two polylinkers of the DP vector constructed as in Example 2, replacing the neo^{r} cassette. The 8-bp cutters on pNEB193 can be used for this purpose, as they are compatible to those on the DP vector. This cloning step can be made easy by adapting the recombination site (att) of the bacteriophage λ to move gene insert between vectors, this recombination being catalysed by a clonase enzyme mix. This vector conversion system may be used to change pNEB193 to a donor vector to clone the target animal gene. Similarly, the DP vector may be converted to a destination vector for the subcloning of the gene after the generation of a deletion in the donor vector.

Although the in vitro transposon system may be initially used, in vivo systems can also be developed where transposition is achieved by bacterial mating. Such systems require the following components: 1) The origin of plasmid transfer (oriT) on the vector; 2) an E. coli strain providing the mini-transposon, the transposase and the transfer functions to drive the conjugational transfer of plasmids containing oriT. The vector containing the target gene will be first transformed into this strain which may be mated to a recipient strain selecting for the antibiotic resistance marker carried by the transposon. Such a system has been developed to mutagenise bacterial genes by Tn5 insertion (Zhang et al, 1993 FEMS Microbiol. Lett, 108:303-310). This can be adapted for the purpose of this invention by two modifications. First, mutant transposase which works in trans need to be generated. Second, two different transposons may be required to eliminate the phenomenon of transposition immunity.

### Example 2

### Modification using the DP Vector System

Method for the assembly of the generic DP knockout vector may be performed by any methods available to the skilled addressee when applied in the manner and combination described. Similarly assembly of an inducible Cre (or FLP) system with the concomitant generation of cell lines stably expressing this inducible recombinase is available to a person familiar to the art (A detailed description of this procedure is generally provided by Bujard at http://www.zmbh.uni-heidelberg.de/Bujard/Homepage.html).

The selection of desired homologous recombination events is based on distinguishing between targeted and random events. In a targeted event recombination occurs with the target DNA sequence and DNA sequence one and two (FIG. 3A; NB the triangles represent loxP sites), resulting in the exclusion of the loxP sites at either end of the vector. Hence ongoing maintenance of the positive- selection of cells expressing the marker DNA will result in survival of the cells. Induction of the recombinase (Cre) and hence the second positive selection event will have no effect on the targeting event. Alternately in a random event (FIG. 3B), one or both ends of the vector will (generally) remain intact leaving two or three functional loxP sites being incorporated into the cells genome. Activation of the inducible recombinase (Cre) within the cell will result in a non-functional marker (in this case the promoter is deleted) and hence will the cell be maintained on the positive-selection conditions will die or be excluded from the selection process.

In some circumstances the recombination event will be inefficient resulting in a relatively low rate of exclusion on non-homologous recombination events. The primer sequence will function in these cases to enable detection of relatively rare events that would be detectable by PCR reactions. Hence at the end of the DP selection process a cell or cells would be continued to be maintained under Cre allowing for ongoing but infrequent rates of recombination to occur—which can be detected by a PCR reaction using primers specific to the Primer Sequencein the exreme left and right regions outside of the loxP site in FIG. 3A) and the First or Second DNA sequence.

### Example 3

### Construction of DP Vector and Insertion of a Target Gene

A double positive selection (DP) is illustrated in FIG. 3. The fact that the whole vector is usually integrated into the chromosome in a non-homologous insertion (random integration) forms the basis of this DP vector. The neo^{r} marker is used here only as an example and other markers are equally suitable including β-geo, hygromycin resistance, zeomycin resistance, HPRT gene and GFP. Also, other recombination systems such as FLP/FRT can be used to replace Cre/LoxP.

The final construct containing the target gene may have the following order: a primer binding sequence—a LoxP site—the short arm of the target gene—a promoter to drive the neo^{r} gene—another LoxP site—the neo^{r} gene—the long arm of the target gene—a third LoxP site—another primer binding site (FIG. 3D). In this vector, the neo^{r} gene may be separated from its promoter by a copy of LoxP. It is known that the separation of the promoter and the gene by a copy of LoxP does not affect the transcription of the gene. After transfection, cells with both a targeted event and a random insertion event will be resistant to neomycin (the first positive selection). However, after expression of the Cre recombinase (under the control of the pTet-on system—Clontech), the cells with a gene-targeted event (FIG. 3B) will be still resistant to neomycin (the second positive selection). By contrast, the cells with a random insertion (FIG. 3C) will become sensitive to neomycin due to deletion between any two LoxP sites, which would eliminate either the promoter or the structural neo^{r} gene or both.

A DP vector for general use may be constructed from pNEB193 (New England Biolabs) which is a pUC19 derivative carrying single sites for three 8-bp cutters in the polylinker. The construction of the DP vector will be achieved by the following procedures (FIG. 3D). A LoxP sequence may be cloned between the EcoR1 and Kpn1 sites at the left side of the polylinker using annealed oligonucleotides, with a Not1 site introduced. Another LoxP sequence may be cloned between the Pst1 and HindIII sites at the right side of the polylinker using annealed oligonucleotides, with the introduction of an Fse1 site. The restriction sites for the 8-bp cutters Not1 and Fse1 will facilitate linearisation of the vector before transfection to animal cells. The neo^{r} gene (without promoter) may then be amplified by PCR from pCl-noe (Promega) and cloned between the BamH1 and Pac1 sites in the middle of the polylinker. Finally, the CMV enhancer/promoter may be amplified from pCl-neo (incorporate LoxP site at the 3′-end) and cloned 5′ to the neo^{r} gene. There are other promoters that are potentially useful including PGK, or β-actin; alternately tissue of cell specific promoters may be utilised that would confer expression in specific cell lines eg protamine promoter in male germ cells. In each step, suitable restriction sites may be incorporated in the oligonucleotides.

On the new DP vector thus constructed, two polylinkers may be derived from the pNEB193 polylinker, separated by the neo^{r} cassette. The first covers the region from Kpn1 to BamH1 with the 8-bp cutter Asc1 whereas the second covers the region between Pac1 and Pme1 with two 8-bp cutters (Pac1 and Pme1). These two polylinkers may be used to clone the short and long arms, respectively, of the target gene.

For the second positive selection in animal cells (to ablate neomycin resistance in non-homologous events) in both systems developed in this study, the timing of Cre expression is important. This controlled Cre expression may be achieved by two means. In transgenic and knockout experiments, chromosomally integrated plasmid DNA may cause undesirable site effects. To overcome this problem, transient expression of the Cre recombinase has been used to remove DNA segments flanked by LoxP sites. Of particular interest is the use of adenovirus vectors expressing Cre (Kanegae et al, 1995; Kaartinen & Nagy, 2001). These vectors rarely integrate into the chromosome and they do not replicate in normal cell lines, because they are replication-defective and can only be propagated in special cell lines providing the replication functions. Furthermore, the transfection efficiency is much higher than plasmid expression vectors (nearly 100% for adenoviruses compared with ˜20% for plasmid expression vectors). NB any method may be utilised to express Cre including transfection of protein or cDNA, microinjection of protein or cDNA.

### Example 4

### Construction of a Double Positive Selection Vector with Two Positive Selectable Markers

This vector is the same as the DP vector in FIG. 3A except that another LoxP site and a promoter-less hygromycin resistance (Hyg^{r}) gene will be present after the Neo^{r} gene. The vector will be used to transfect rat cells selecting for neomycin resistance. After a targeted event, expression of the Cre recombinase using the adenovirus-Cre will delete the Neo^{r} gene, allowing the expression of the Hyg^{r} gene, conferring the cells hygromycin resistance. In a random integration event with the two outside LoxP sites present, expression of Cre recombinase will delete the promoter or the Hyg^{r} gene (or both), rendering the cells sensitive to hygromycin. Such a DP vector will be constructed by inserting a LoxP-Hyg^{r} fragment at the Pacl site after the Neo^{r} gene in FIG. 3D. The vector is shown in FIG. 4.

### Example 5

### Development of a Transposon-Mediated Procedure to Generate Deletions

a) Oligonucleotide primers.

Oligonucleotide primers for PCR reactions to amplify DNA segments to construct the various transposons are shown below.

| | |
|---|---|
| Mu1-1: | |
| CTGGGTACCAGATCTGAAGCGGCGCACGAAAAACGCGAAAGCGTTTCACG | (SEQ ID NO:1) |
| ATAAATGCGAAAACATTCAAATATGTATCCGCTC | |
| Mu1-2: | |
| CTGCCCGGGATAACTTCGTATAATGTATGCTATACGAAGTTATCCTG | (SEQ ID NO:2) |
| TCTCTTGATCGATCTTTGC | |
| Mu1-3: | |
| CTGGTCGACGCTAAGGAAGCTAAAATGGAG | (SEQ ID NO:3) |
| Mu1-4: | |
| CTGAAGCTTAGATCTGAAGCGGCGCACGAAAAACGCGAAAGCGTTTCACG | (SEQ ID NO:4) |
| ATAAATGCGAAAACGTCAATTATTACCTCCACG | |
| Mu2-1: | |
| CTGGGTACCAGATCTGAAGCGGCGCACGAAAAACGCGAAAGCGTTTCACG | (SEQ ID NO:5) |
| ATAAATGCGAAAACTTCTCATGTTTGACAGCTTATC | |
| Mu2-2: | |
| CTGCTCGAGCCGCAAGAATTGATTGGCTCC | (SEQ ID NO:6) |
| Mu2-Neo-1 | |
| CTGCTCGAGATAACTTCGTATAGCATACATTATACGAAGTTATAGGA | (SEQ ID NO:7) |
| GCCGCCACCATGATTGAACAAGATGGATTGC | |
| Mu2-Neo-2 | |
| CTGTCTAGATCTGAAGCGGCGCACGAAAAACGCGAAAGCGTTTCAC | (SEQ ID NO:8) |
| GATAAATGCGAAAACACACAAAAAACCAACACACAG | |
| Mu2-HygGFP-1: | |
| CTGCTCGAGATAACTTCGTATAGCATACATTATACGAAGTTATAGGA | (SEQ ID NO:9) |
| GCCGCCACCATGAAAAAGCCTGAACTCACCGCG | |
| Mu2-HygGFP-2: | |
| CTGAGATCTTACTTGTACAGCTCGTCCATG | (SEQ ID NO:10) |
| Mu2-geo-1: | |
| CTGCTCGAGATAACTTCGTATAGCATACATTATACGAAGTTATAGGA | (SEQ ID NO:11) |
| GCCGCCACCATGGAAGATCCCGTCGTTTTACAACGTCG | |
| GeoSacBglXba: | |
| CTGTCTAGAGAGAGATCTTCTGAGCTCGTTATCGCTATGAC | (SEQ ID NO:12) |
| SacGeo: | |
| CTGGAGCTCCTGCACTGGATGGTG | (SEQ ID NO:13) |
| Mu2-geo-2: | |
| CTGAGATCTCAGAAGAACTCGTCAAGAAGG | (SEQ ID NO:14) |
| Mu2-polyA1: | |
| CTGGGATCCGAGCAGACATGATAAGATAC | (SEQ ID NO:15) |
| Mu2-polyA-2: | |
| CTGTCTAGATCTGAAGCGGCGCACGAAAAACGCGAAAGCGTTTCACGATA | (SEQ ID NO:16) |
| AATGCGAAAACTTACCACATTTGTAGAGGTTTTACTTGC | |
| Mu1CamEndOutward: | |
| CGTGGAGGTAATAATTGACG | (SEQ ID NO:17) |
| HPRTexon4F: | |
| CTTGCACTCACTAGGCAAGC | (SEQ ID NO:18) |
| HPRTexon5F: | |
| GGACCCTTCTGAGTTCTAATAAGC | (SEQ ID NO:19) |
| HPRTexon6F: | |
| CCACTGCTTGCTTAGAACCAG | (SEQ ID NO:20) |
| HPRTexon7-9F: | |
| GTTGCATTTCAGTGTGGGTG | (SEQ ID NO:21) |

b) PCR.

PCR was carried out using a GeneAmp PCR System 2700 (Applied Biosystem). Template DNA (10-100 ng) was amplified in 50 μl reaction mixture containing 200 μM of each dNTP, 20 pmol of each primer, 1.25 U of Taq DNA polymerase in 1×PCR buffer containing MgCl₂ (Fischer Biotech). The reaction was carried out for 30 cycles under the following conditions: denaturation, 30 s at 94° C.; primer annealing, 30 s at 55° C.; primer extension, 150 s at 72° C. The denaturation step in the initial cycle was extended to 150 s and the primer extension step in the final cycle was extended to 570 s.

c) DNA cloning.

Because of the relatively low efficiency of cloning PCR products with restriction sites introduced by incorporating such sites at the 5′-end of the primers (Jung et al. Nucleic Acids Res. 18: 6156, 1990), the PCR products were first cloned to a vector by blunt ligation (Zhang et al. Biochem. Biophys. Res. Commun. 242: 390-395, 1998). To do this, the PCR product was treated with Klelow fragment as described by Obermaier-Kusser et al (Biochem. Biophys. Res. Commun. 169: 1007-1015, 1990) to eliminate artifactually polymeraised deoxyadenylic acid at the 3′-end. The product was then gel purified using the Gel Purification Kit (Qiagen) and cloned to the plasmid vector digested with a blunt end restriction enzyme (Zhang et al. FEBS Lett. 297: 34-38, 1992). The insert was sequenced, when considered necessary, and subcloned to suitable vectors by digestion with compatible restriction enzymes and ligation.

d) In vitro transposition.

The transposon was released from the bacterial vector by digesting with BgIII and gel purified. The in vitro transposition reaction (20 μl) contained 20 ng mini-Mu transposon (BgIII fragment), 400 ng target plasmid DNA and 0.22 μg of MuA transposase in 1× transposition buffer (FinnZyme). The reaction was carried out for 1 h at 37° C., followed by incubation at 75° C. for 10 min to inactivate the transposase. The reaction mixture was used to tramsform E. coli DH5α, or to electroporate E. coli DH10β selecting for the appropriate antibiotic resistance marker.

1. Construction of Mini-Mu Transposon 1

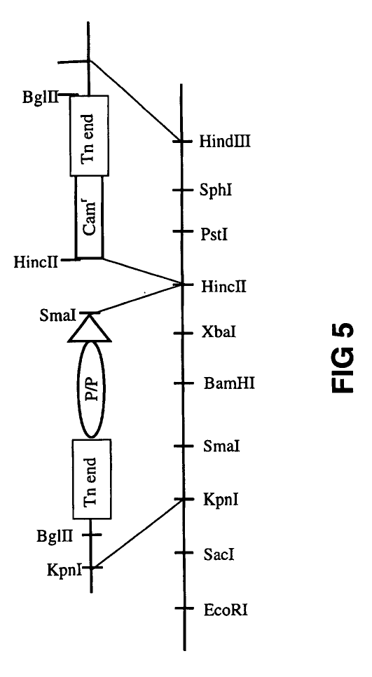

The prokaryotic/eukaryotic double promoter (P/P) was amplified by PCR from the Clontech vector pEGFP-N1 using primers Mu1-1 and Mu1-2, incorporating the Mu transposon end at the 5′-end and a LoxP sequence at the 3′-end. A KpnI site and a BgIII site were introduced at the 5′-end and a Smal site at the 3′-end. This product was cloned at the SmaI site of pUC9 to form pCO6. The chloramphenicol resistance gene (Cam^{r}) was amplified by PCR from pACYC184 using primers Mu1-3 and Mu1-4, incorporating the Mu transposon end at the 3′-end. A HincII site were introduced at the 5′-end, and a BgIII site and a HindIII site at the 3′-end. This product was cloned between the HinII sites of pUC7 to form pCO7. The Cam^{r} insert was released from pCO7 by digesting with HincII and HindIII and cloned between the HincI and HindIII sites of pUC19 to form pCO9. The P/P insert was released from pCO6 by digesting with KpnI and SmaI and cloned between the KpnI and HincI sites of pCO9. This completes the construction of Mini-Mu transposon 1 which contains the double promoter P/P and Cam^{r} separated by a LoxP sequence, the whole gene construct flanked by transposon ends. The vector carrying this transposon was designated as pCO10 (FIG. 5). Digestion with BgIII would release the transposon from the vector.

2. Testing of Mini-Mu Transposon 1

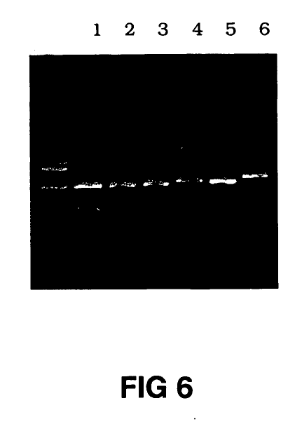

Mini-Mu transposon 1 (Mu1-Cam) was tested for its ability to transpose in vitro, with pUC7 as the target DNA molecule, selecting for chloramphenicol resistance. When 50 choloramphenicol resistant colonies were patched on ampicillin plate, 13 (26%) were found to be sensitive to ampcillin, indicating that the transposon had inserted to and inactivated the Amp^{r} gene. Considering the proportion of the Amp^{r} gene on pUC7 (30%), the insertion of Mu1 on this plasmid was random. This was further confirmed by restriction digestion. Three Amp^{r} colonies and three Amp^{s} colonies were selected and plasmid DNA isolated. The DNA was digested with Sspl which cuts once in pUC7 and twice in the transposon. Different patterns were observed (FIG. 6), suggesting the random nature of the transposon insertion on pUC7. In FIG. 6, Lanes 1-3 are pUC7 with the transposon inserted to the Amp^{r} gene and Lanes 4-6 are pUC7 with the transposon inserted outside of the Amp^{r} gene.

3. Construction of Mini-Mu Transposon 2:

Three different versions of Mini-Mu transposon 2 were constructed. The 5′-end was the same for all three versions which was the transposon end and the bacterial tetracycline resistance gene (Tet^{r}). This was constructed as follows. The Tet^{r} gene was amplified by PCR from pBR322 with primers Mu2-1 and Mu2-2, incorporating KpnI-BgIII-ransposon end at the 5′ side and a XhoI site at the 3′-end. This product was cloned at the HincII site of pNEB193 to form pCO19. The completion of the three versions of Mini-Mu transposon 2 was as follows.

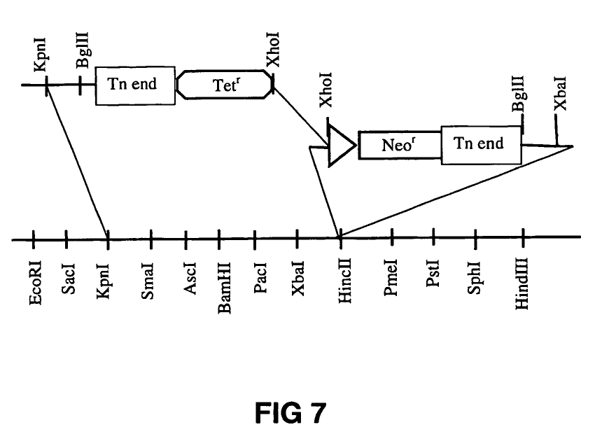

a) Mu2-Neo. This transposon has the neomycin resistance gene (Neo^{r}) downstream of the Tet^{r} gene. The Neo^{r} gene was amplified from pCl-neo (Promega) with primers Mu2-Neo-1 and Mu2-Noe-2, incorporating XhoI-LoxP at the 5′ end and the transposon end-BgIII-XbaI at the 3′-end. This product was cloned at the HincII site of pNEB193 in such a way that the XhoI site on the insert was toward the KpnI site on the vector to form pCO18. The insert of pCO19 was excised using KpnI and XhoI, and cloned between the KpnI and XhoI sites of pCO18. This vector carrying the transposon Mu2-Neo was designated as pCO20 (FIG. 7).

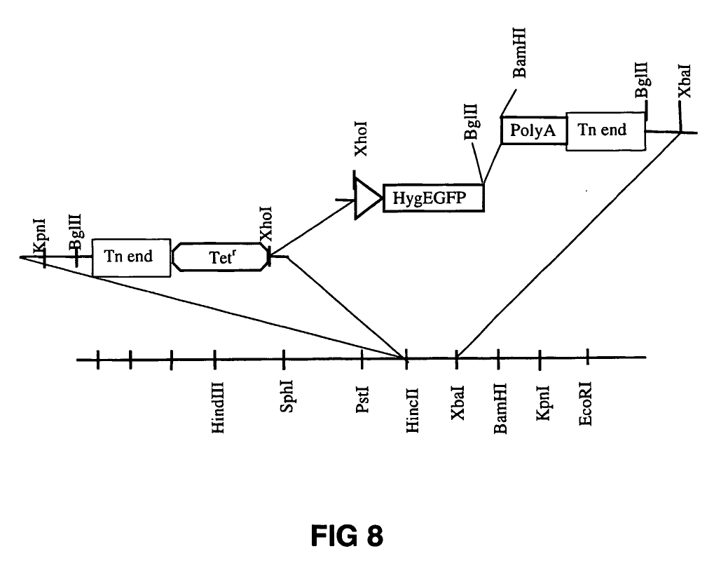

b) Mu2-HygEGFP. This transposon has the hygromycin resistance-EGFP fusion gene (HygEGFP) downstream of the Tet^{r} gene. The coding region of the HygEGFP gene was amplified from pHygEGFP (Clontech) with primers Mu2-HygEGFP-1 and Mu2- HygEGFP-2, incorporating XhoI-LoxP at the 5′ end and a BgIII site at the 3′-end. This product was cloned at the HincII site on pNEB193 to form pCO15. The sequence containing the SV40 polyA signal was amplified from the same plasmid using primers Mu2-PolyA-1 and Mu2-PolyA-2, incorporating a BamHI site at the 5′ end and the transposon end-BgIII-XbaI at the 3′-end. This product was also cloned at the HincII site on pNEB193 to form pCO16. The polyA insert was excised with BamHI and XbaI and cloned between the BgIII and XbaI sites of pCO15 to form pCO21. The HygEGFP gene including the polyA was cut out with XhoI and XbaI and cloned between the XhoI and XbaI sites of pCO19. This vector carrying the transposon Mu2-HygEGFP was designated as pCO25 (FIG. 8).

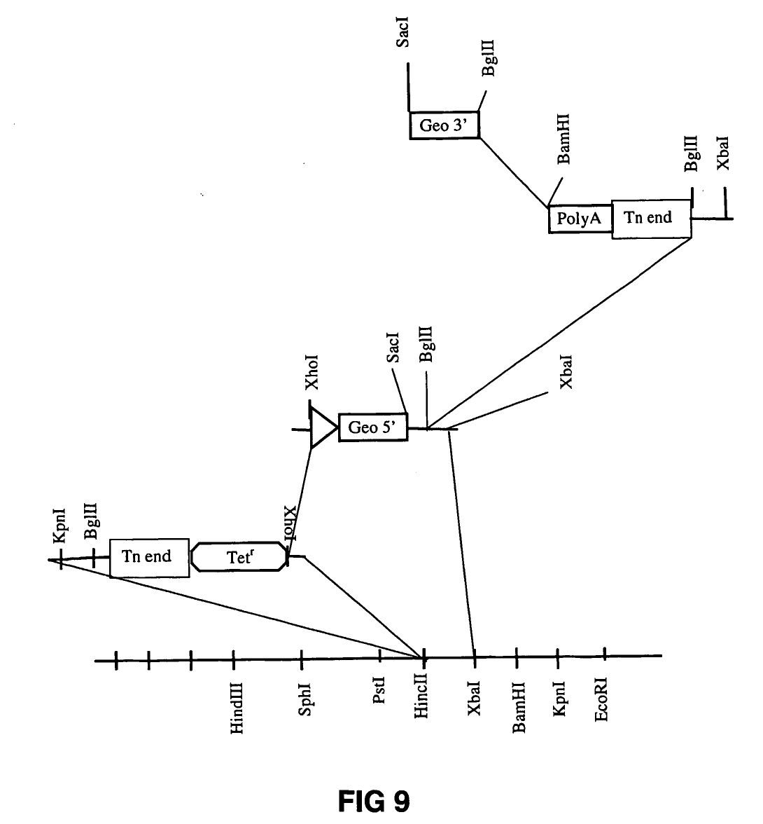

c) Mu-2-β-geo. This transposon has the β-galactosidase-neomycin resistance fusion gene (β-geo) downstream of the Tet^{r} gene. Because the coding region of the β-geo gene is about 4 kb and could not be amplified efficiently under our PCR conditions, the gene was amplified as two fragments and subsequently joined together taking advantage of the SacI site in the middle of the gene. The 5′ half of the gene was amplified from pHβAclβgeo (E. Stanley, Pers. Commun.) with primers Mu2-geo-1 and GeoSacBgIXba, incorporating XhoI-LoxP at the 5′ end and a BgIII-XbaI sites at the 3′-end after the natural SacI site. This product was cloned at the HincI site of pNEB193 by blunt end ligation (to form pCO22) and subsequently to pCO4 (this vector does not have SacI sites, see below) using PacI and PmeI (to form pCO40). The 3′ half of the β-geo gene was amplified with primers SacGeo and Mu2-geo-2, with the natural SacI site at the 5′ end and incorporating a BgIII site at the 3′-end. This product was cloned at the HincI iste of pUC7 to form pCO 13. The BamHI-BgIII fragment containing the polyA signal from pCO16 was then cloned at the BgIII site of pCO13 to form pCO41. The geo2::PolyA part was released by digesting with SacI and BgIII and cloned between the SacI and BgIII sites on pCO40 to form pCO42. This resulted in a complete β-geo gene with the polyA signal followed by a transposon end. This construct was excised with XhoI and XbaI and cloned between the XhoI and XbaI sites of pCO19. This vector carrying the transposon Mu2-β-geo was designated as pCO43 (FIG. 9).

### Example 6

### Transposon Mutagenesis of the Rat HPRT Gene

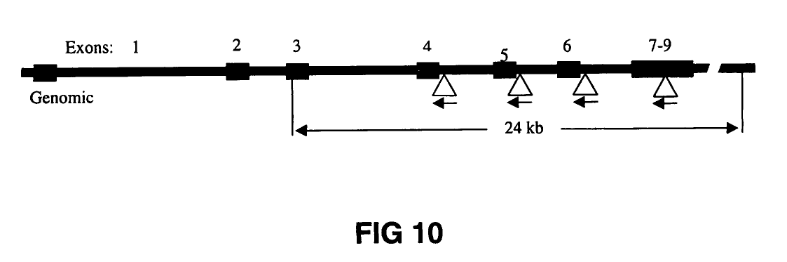

A 24 kb XhoI fragment containing part of exon 3 and exons 4-9 (see FIG. 10) of the rat HPRT gene was cloned from a PAC clone into the Sall site of pNEB193 to form pCO28. This clone was used as the target for transposition by mini-Mu transposon 1 with the selection of chloramphenicol resistance. The oligonucleotide Mu1CamEndOutward, which is located at the end of Mini-Mu transposon 1 with the 3′-end pointing outward, was combined with each of four primers for PCR. They were HPRTexon4F, HPRTexon5F, HPRTexon6F, HPRTexon7-9F. When 49 Cam^{r} colonies were screened, 1 to 3 colonies gave a PCR product within 1 kb for each PCR reaction, i.e. 2-6%. The predicted probability of the transposon to insert in any 1 kb region at one orientation on a 27 kb plasmid (including the vector) is 2%. Considering the small number of colonies screened, the obtained percentage is acceptable. One such colony for each PCR was selected. They represented transposon insertions whose approximate locations are shown by the triangles in the FIG. 10, with the direction of Cam^{r} gene transcription indicated by an arrow. They have the desirable orientation of the transposon inserts and transposition of mini-Mu2-Neo into them may be conducted similarly.

### Example 7

### Construction of DP Vector and Insertion of a Target Gene

a) Oligonucleotides:

| | |
|---|---|
| Oligo1: | |
| AATTGCGGCCGCATAACTTCGTATAGCATACATTATACGAAGTTATG | (SEQ ID NO:22) |
| GTAC | |
| Oligo2: | |
| CATAACTTCGTATAATGTATGCTATACGAAGTTATGCGGCCGC | (SEQ ID NO:23) |
| Oligo3: | |
| GATAACTTCGTATAGCATACATTATACGAAGTTATGGCCGGCC | (SEQ ID NO:24) |
| Oligo4: | |
| AGCTGGCCGGCCATAACTTCGTATAATGTATGCTATACGAAGTTATC | (SEQ ID NO:25) |
| TGCA | |
| OligoNeo1: | |
| CTGGGATCCGCCGCCACCATGATTGAACAAGATGGATTGC | (SEQ ID NO:26) |
| OligoNeo2: | |
| CTGTTAATTAACACACAAAAAACCAACACACAG | (SEQ ID NO:27) |
| OligoCMVProm1: | |
| CTGGGATCCTCAATATTGGCCATTAGCC | (SEQ ID NO:28) |
| OligoCMVProm2: | |
| CTGAGATCTATAACTTCGTATAATGTATGCTATACGAAGTTATGATCT | (SEQ ID NO:29) |
| GACGGTTCACTAAACG | |
| OligoPGKProm1: | |
| CTGGGATCCTACCGGGTAGGGGAGGCG | (SEQ ID NO:30) |
| OligoPGKProm2: | |
| CTGAGATCTATAACTTCGTATAATGTATGCTATACGAAGTTATGTCG | (SEQ ID NO:31) |
| AAAGGCCCGGAGATGAG | |

b) PCR and cloning.

These were carried out the same as described above in Example 5 with the following addition. When two oligonucleotides were to be annealed and cloned, 50 pmol of each primer were mixed in a final volume of 10 μl and incubated at 95° C. for 5 min in a heating block. The heating block was then turned off and the sample allowed to cool down slowly to room temperature in the heating block. The vector was digested with two enzymes without compatible ends and the annealed oligonucleotides cloned into the vector.

1. Construction of DP vectors.

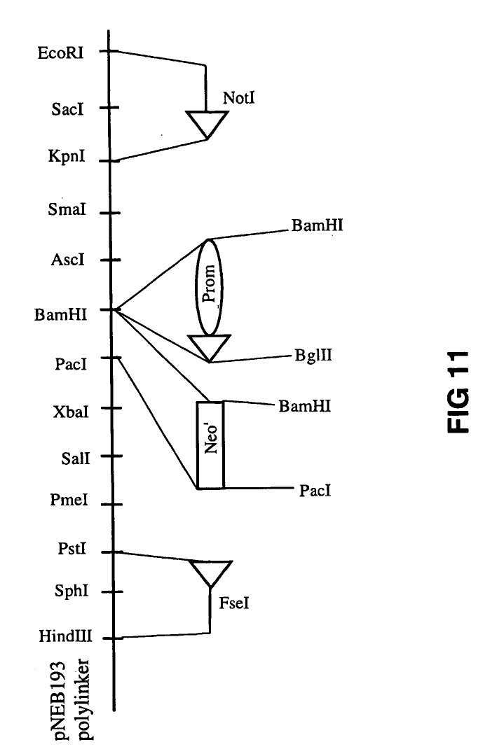

Two versions of DP vector were constructed, one with the CMV promoter and the other with the PGK promoter, both driving the expression of Neo^{r}. Oligo1 and oligo2 were annealed and cloned between the EcoRI and KpnI sites of pNEB193 to form pCO3. This introduced a NotI site and a LoxP sequence and, at the same time, destroyed the EcoRI site. Oligo3 and oligo4 were then annealed and cloned between the PstI and HindIII sites of pCO3 to form pCO4. This introduced a LoxP sequence and an FseI site and, at the same time, destroyed the HindIII site. The neomycin resistance gene (Neo^{r}) was amplified from pCl-neo with primers OligoNeo1 and OligoNeo2 and cloned between the HincI sites of pUC7 to form pCO2. The Neo^{r} gene was then released with BamHI and PacI and cloned between the BamHI and PacI sites of pCO4 to form pCO5. The CMV promoter was amplified from pCl-neo with primers OligoCMVProm1 and OligoCMVProm2 and cloned between the HincI sites of pUC7 to form pCO1. Similarly, the PGK promoter was amplified from pKO Scrambler NTKY-1906 with primers OligoPGKProm1 and OligoPGKProm2 and cloned between the HincII sites of pUC7 to form pCO12. These two promoters were released from the respective vectors with BamHI and BgIII and cloned at the BamHI site of pCO5 to form the two versions of the DP vector, respectively. The DP vector with the CMV promoter was designated as pCO8 and the one with the PGK promoter as pCO14 (FIG. 11).

2. Testing the DP vectors in E coli.

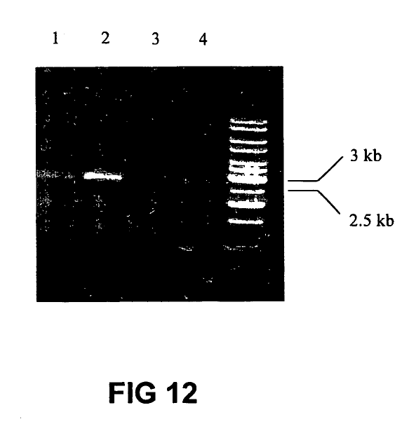

The two DP vectors, pCO8 and pCO14, were transformed into an E. coli strain expressing the Cre recombinase. Plasmid DNA was extracted and linearised by Not1 digestion. In FIG. 12, Lanes 1 and 2 are pCO8 whereas Lanes 3 and 4 are pCO14. As judged by the size of the plasmid (2.7 kb), both the neomycin resistance gene and the promoter were deleted. This indicates that the sequences flanked by LoxP sites on these vectors could be efficiently removed by recombination.

### Example 8

### Construction of a Double Positive Selection Vector with Two Positive Selectable Markers

a) Oligonucleotide primers:

| | |
|---|---|
| PvulLoxHyg: | |
| CTGCGATCGATAACTTCGTATAGCATACATTATACGAAGTTATGCCG | (SEQ ID NO:32) |
| CCACCATGAAAAAGCCTG | |
| HygPacl: | |
| CTGTTAATTAAGATCTATAGATCATGAGTGG | (SEQ ID NO:33) |

b) PCR and Cloning.

These were carried out the same as described in Examples 5 and 6.

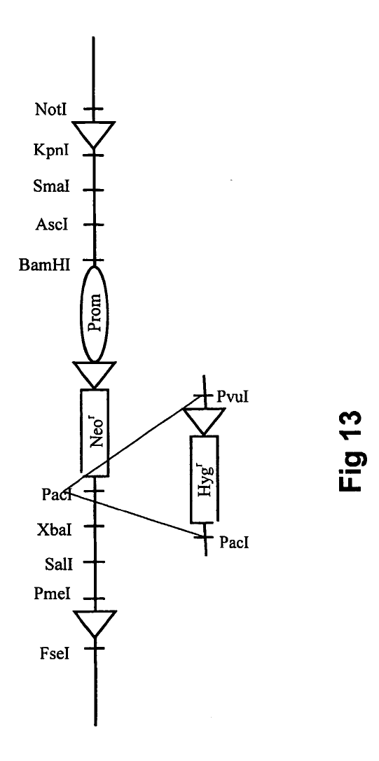

1. Construction of the DP vector (FIG. 13).

The hygromycin resistance gene (Hyg^{r}) was amplified from pPGKHyg with primers PvuILoxHyg and HygPacI, incorporating a PvuI site and a LoxP sequence at the 5′-end and a PacI site at the 3′-end. The product was cloned at the HincII site of pNEB193 to form pCO17. The Hyg^{r} gene was released by complete digestion with PacI followed by partial digestion with PvuI (because there is an internal PvuI site on the gene) and cloned at the PacI site of both versions of the DP vector (pCO8 and pCO14) to form pCO26 (CMV promoter) and pCO27 (PGK promoter).

### Example 9

### Construction of Knockout Vectors for the Rat HPRT Gene

a) Oligonucleotide primers:

| | | |
|---|---|---|
| HPRTexon7-9F: | GTTGCATTTCAGTGTGGGTG | (SEQ ID NO:34) |
| HPRTexon7-9R: | AGGCTGCCTACAGGCTCATA | (SEQ ID NO:35) |

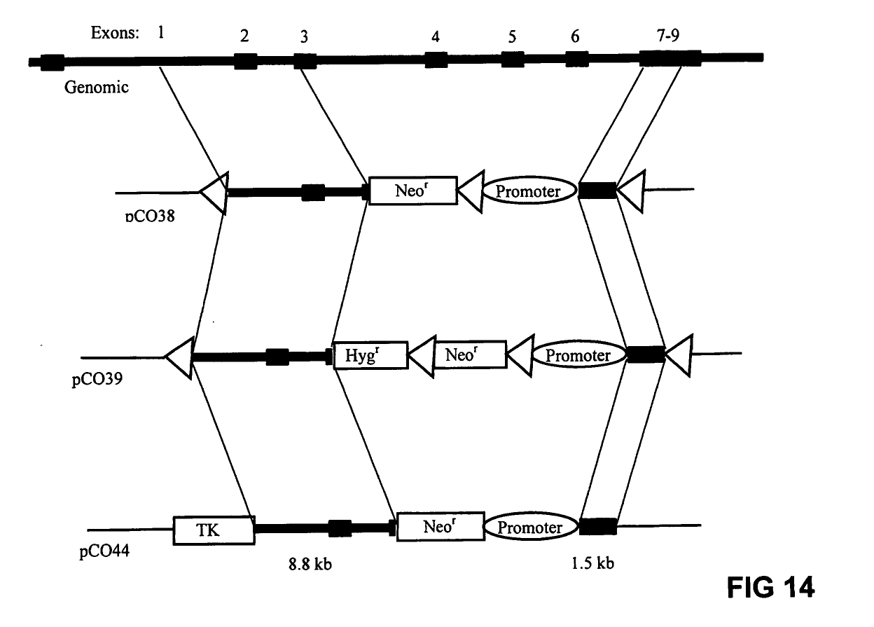

In order to validate the DP vectors, the rat HPRT gene was selected as the target to be knocked out. The short arm was a PCR product amplified from the a PAC clone using primers HPRTexon7-9F and HPRTexon7-9R. This product was cloned between the HincII sites of pUC7 to form pCO30. The insert was released with BamHI and cloned at the BamHI site of two DP vectors pCO14 and pCO27 to form pCO33 and pCO34, respectively. To compare the targeting efficiency with the traditional positive/negative selection vector, the short arm was also clone a at the BamHl site of pKO Scrambler NTKY-1906 to form pCO32. The long arm selected was an 8.8 kb XhoI fragment from intron 1 to exon 3. This was cloned from a PAC clone to the SaII site of pCO33 (to form pCO38) and pCO34 (to form pCO39), and at the XhoI site of pCO32 (to form pCO44). The three targeting constructs are schematically illustrated FIG. 14 (the figure is not drawn to scale).

### Example 10

### Construction of a Vector with Floxed β-geo

a) Primers:

| | |
|---|---|
| Kpn-geo-1: | |
| CTGGGTACCGCCGCCACCATGGAAGATCCCGTCGTTTTACAACGTC | (SEQ ID NO:36) |
| G | |
| GeoSacBglXba | |
| CTGTCTAGAGAGAGATCTTCTGAGCTCGTTATCGCTATGAC | (SEQ ID NO:37) |
| Kpn-PGK-1: | |
| CTGGGTACCACCGGGTAGGGGAGGCG | (SEQ ID NO:38) |
| MuEndEcoSwaPGK-2: | |
| CTGGGTACCGTCGAAAGGCCCGGAGATGAG | (SEQ ID NO:39) |
| Mu2-polyA1: | |
| CTGGGATCCGAGCAGACATGATAAGATAC | (SEQ ID NO:40) |
| PolyA-R: | |
| CTGAGATCTGGTACCTTACCACATTTGTAGAGGTTTTACTTGC | (SEQ ID NO:41) |

In order to test the efficiency of LoxP recombination in mammalian cells, a vector containing β-geo flanked by LoxP sites was constructed. The vector is integrated to the genome of rat cells which will then be infected by an adenovirus vector transiently expressing the Cre recombinase. The percentage of cells which have lost the β-geo gene as determined by X-GaI staining represents the efficiency of Cre-mediated LoxP recombination.

1. Construction of the targeting vector.

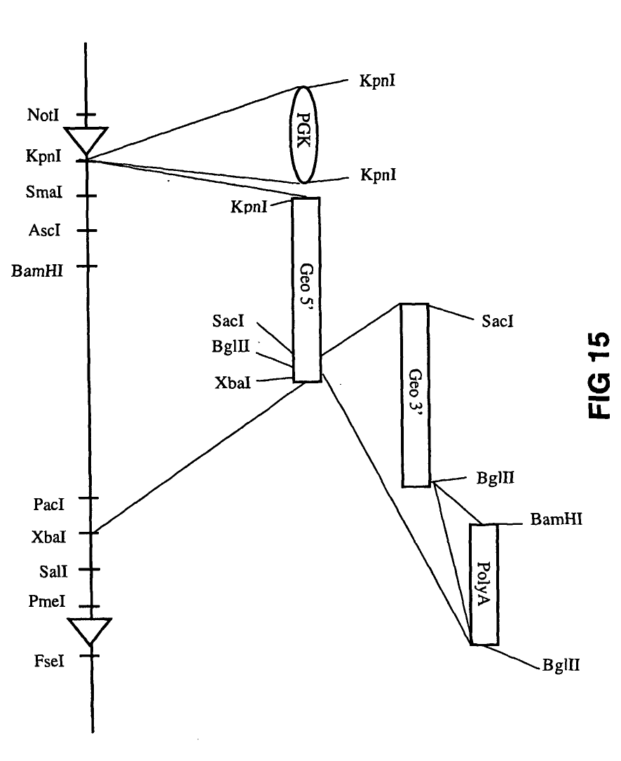

As for the construction of Mini-Mu2-β-geo described in Example 5, the β-geo gene was amplified as two fragments and subsequently joined together taking advantage of the SacI site in the middle of the gene. The 5′ half of the gene was amplified with primers Kpn-geo-1 and GeoSacBgIXba, incorporating Kpnl at the 5′ end and a BgIII-XbaI sites at the 3′-end after the natural SacI site. This product was cloned at the HincI site of pNEB193 by blunt end ligation (to form pCO45) and subsequently to ppCO4 (this vector has LoxP flanking the polylinkers) using KpnI and XbaI (to form pCO46). The PGK promoter was amplified with primers Kpn-PGK-1 and MuEndEcoSwaPGK-2, introducing a KpnI site at each end of the promoter. This product was cloned at the HincII site of pNEB193 by blint end ligation to form pCO47. The insert was released with KpnI and cloned at the KpnI site of pCO46 to form pCO48. The sequence containing the polyA signal was amplified from pHygEGFP using primers Mu2-PolyA-1 and PolyA-R, incorporating a BamHI site at the 5′ end and KpnI-BgIII sites at the 3′-end. This product was cloned at the HincI site on pNEB193 to form pCO49. The polyA insert was excised with BamHI and BgIII and cloned at the BgIII site of pCO13 to form pCO50. The geo2::PolyA part was released from pCO50 by digesting with SacI and BgIII and cloned between the SacI and BgIII sites on pCO48 to form pCO51 (FIG. 15).

### Example 11

### Design of a Southern Strategy to Verify HPRT Knockout

a) Primers

| | | |
|---|---|---|
| HPRTSouthern1 | GTACTCTGTAGTCCAGGCTG | (SEQ ID NO:42) |
| HPRTSouthern2 | CAAGTCTTTCAGTCCTGCAG | (SEQ ID NO:43) |
| HPRTSouthern3 | GAATAGTCTAAAGCGCTCAG | (SEQ ID NO:44) |
| HPRTSouthern4 | GCTAAGAGAAAGCCATGTTCTC | (SEQ ID NO:45) |

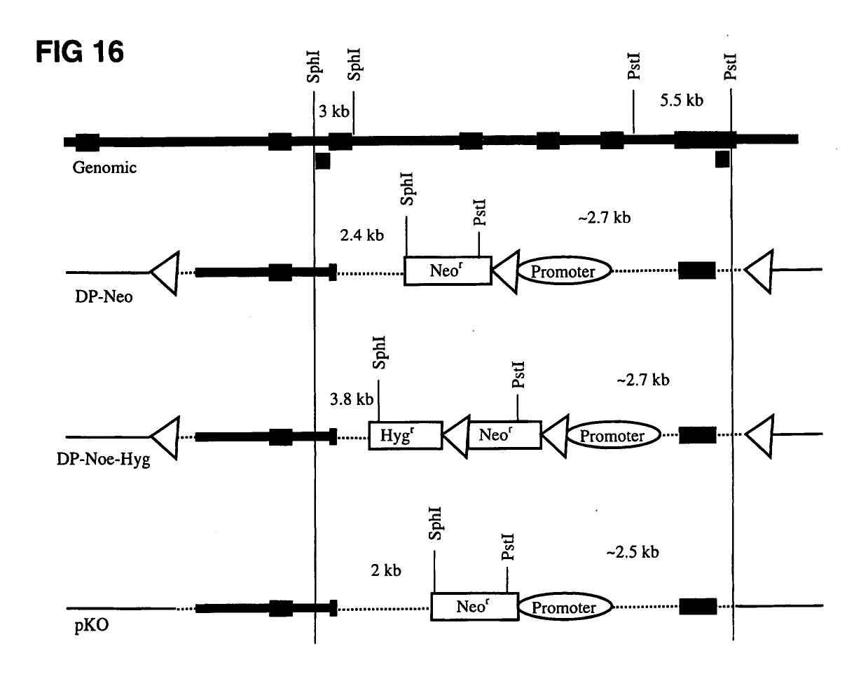

Based on the structures of the three HPRT targeting vectors described above, a Southern hybridization strategy was designed to verify the knockout of the HPRT gene. This strategy is shown below in (FIG. 16 the figure is not drawn to scale with the emphasis on the alignment of the long and short arms).

A 404 bp fragment of the HPRT gene (whose location is represented by the black square at the left) was amplified with primers HPRTSouthern1 and HPRTSouthern2 and cloned between the BamHI sites of pUC7 to form pCO51. When used as a probe, this will hybridise a 3 kb SphI fragment from wild type genomic DNA. The sizes of the fragments hybridized will be 2.4 kb, 3.8 kb and 2 kb, respectively, for knockouts generated with PD-Neo, PD-Neo-Hyg and pKO.

A 414 bp fragment of the HPRT gene (whose location is represented by the black square at the right) was amplified with primers Southern3 and HPRTSouthern4 and cloned between the BamHl sites of pUC7 to form pCO52. When used as a probe, this will hybridise a 5.5 kb PstI fragment from wild type genomic DNA. The sizes of the fragments hybridized will be 2.7 kb, 2.7 kb and 2.5 kb, respectively, for knockouts generated with PD-Neo, PD-Neo-Hyg and pKO.

Finally it is to be understood that various other modifications and/or alterations may be made without departing from the spirit of the present invention as outlined herein.

1-56. (canceled)

57. A method of preparing a targeting construct for use in a targeting vector, wherein said targeting vector is capable of modifying a target DNA sequence, said method comprising the steps of:
(a) obtaining a copy of the target DNA sequence in vitro;
(b) inserting a first DNA sequence comprising a first transposon sequence and a first DNA recombination sequence at a first site in the copy of the target DNA sequence;
(b) inserting a second DNA sequence comprising a second transposon sequence and a second DNA recombination sequence at a second site in the copy of the target DNA sequence; and
(c) inducing a recombination event between said first and second recombination sequences to delete a portion of the copy of the target DNA sequence.

58. A method according to claim 57 wherein the inserted DNA sequence includes a mini-transposon.

59. A method according to claim 58 wherein the transposon is selected from the group consisting of Mu1-Cam, Mu2-Neo, Mu2-Hyg EGFP andMu2-ss-geo.

60. A method according to claim 57 wherein the recombination sequence is at least one sequence that is selected from the group consisting of a 1oxp sequence and an inverted repeat sequence (FRT) that is under the influence of a recombinase.

61. A method according to claim 60 wherein the recombinase is selected from the group consisting of Cre, FLP and a member of the intergrase family of recombinases.

62. A method according to claim 61 wherein the member of the integrase family of recombinases is selected from the group consisting of Gln, Hin and resolvase.

63. A method according to claim 57 wherein the recombination event is mediated by a Cre-loxP recombinase system.

64. A method according to claim 57 wherein the transposon sequence comprises at least one of a selectable marker encoding sequence and a promoter sequence.

65. A method according to claim 64 wherein the selectable marker encoding sequence is selected from the group consisting of an antibiotic resistance encoding sequence and an enzyme encoding sequence.

66. A method according to claim 65 wherein the antibiotic resistance resistance encoding sequence encodes a selectable marker that is selected from the group consisting of a chloramphenicol resistance marker, a tetracycline resistance marker, and a neomycin resistance marker.

67. A method according to claim 65 wherein the enzyme encoding sequence encodes a selectable β-geo marker.

68. A method according to claim 57 wherein the first and second DNA sequences are inserted sequentially into the target DNA.

69. A method according to claim 57 wherein the step of inducing the recombination event comprises induction by a Tet-on system or by an ecdysone inducible system.

70. A pre-targeting construct for use in creating deletions in a target DNA sequence, said construct comprising:
a copy of a target DNA sequence; and
at least two transposon units each comprising a recombination sequence, wherein said transposon units are inserted and positioned within the copy of the target DNA so that upon a recombination event between the recombination sequences, a portion of the copy of target DNA is deleted.

71. A pre-targeting construct according to claim 70 wherein at least one transposon unit comprises a mini-mu transposon unit.

72. A pre-targeting construct according to claim 70 wherein at least one transposon unit is selected from the group consisting of Mu1-Cam, Mu2-Neo, Mu2Hyg EGFP andMu2-p-geo.

73. A pre-targeting construct according to claim 70 wherein at least one recombination sequence is selected from the group consisting of a 1oxp sequence and an inverted repeat sequence (FRT) that is under the influence of a recombinase.

74. A pre-targeting construct according to claim 73 wherein the recombinase is selected from the group consisting of Cre, FLP and a member of the intergrase family of recombinases.

75. A pre-targeting construct according to claim 74 wherein the member of the integrase family of recombinases is selected from the group consisting of Gln, Hin and resolvase.

76. A pre-targeting construct according to claim 70 wherein the recombination event is mediated by a Cre-loxP recombinase system.

77. A pre-targeting construct according to claim 70 wherein at least one transposon unit comprises a selection marker.

78. A pre-targeting construct according to claim 77 wherein the selection marker is selected from the group consisting of an antibiotic resistance marker and an enzyme marker.

79. A pre-targeting construct according to claim 78 wherein the antibiotic resistance marker comprises a sequence that encodes a selectable marker that is selected from the group consisting of a chloramphenicol resistance marker, a tetracycline resistance marker, a neomycin resistance marker and a β-geo marker.

80. A targeting construct prepared by a method according to claim 57.

81. A double positive (DP) vector for modifying a target DNA sequence contained in the genome of a cell, said DP vector comprising:
a first DNA sequence comprising a first homologous vector DNA sequence that is capable of homologous recombination with a first region of said target DNA sequence;
a second DNA sequence comprising a positive selection marker DNA sequence that is capable of conferring a positive selection characteristic in said cells;
a third DNA sequence that supports high-efficiency DNA recombination in the presence of a site specific recombinase and which is contained within the positive selection marker DNA sequence;
a fourth DNA sequence comprising a second homologous vector DNA sequence that is capable of homologous recombination with a second region of said target DNA sequence; and
a fifth DNA sequence which directs site specific recombination with the third sequence, but which is substantially incapable of homologous recombination with said target DNA sequence,
wherein the spatial order of said DNA sequences in said DP vector is: said first DNA sequence, said second DNA sequence containing the third DNA sequence, said fourth DNA sequence, and said fifth DNA sequence,
and wherein the vector is capable of modifying said target DNA sequence by homologous recombination of said first homologous vector DNA sequence with said first region of said target sequence and homologous recombination of said second homologous vector DNA sequence with said second region of said target DNA sequence.

82. A DP vector according to claim 81 wherein the first and fourth DNA sequences comprise portions of DNA which are each substantially homologous to a corresponding portion in a first and second region of the target DNA.

83. A DP vector according to claim 81 wherein the first and fourth DNA sequences hybridize under stringent hybridization conditions to a first and second region, respectively, of the target DNA.

84. A DP vector according to claim 81 wherein the first homologous vector DNA sequence of the first DNA sequence and the second homologous vector DNA sequence of the fourth DNA sequence do not exhibit sequence polymorphisms.

85. A DP vector according to claim 81 wherein the positive selection marker DNA sequence encodes a marker that is selected from the group consisting of a drug resistance gene product, a fluorescent marker and a bioluminescent markers.

86. A DP vector according to claim 81 wherein the positive selection marker DNA sequence is positioned between the first and fourth DNA sequences.

87. A DP vector according to claim 81 wherein the third DNA sequence is selected from the group is selected from the group consisting of a loxP sequence and an inverted repeat sequence (FRT) that is under the influence of a recombinase.

88. A DP vector according to claim 87 wherein the recombinase is selected from the group consisting of Cre, FLP and a member of the intergrase family of recombinases.

89. A DP vector according to claim 88 wherein the member of the integrase family of recombinases is selected from the group consisting of Gln, Hin and resolvase.

90. A DP vector according to claim 81 wherein the third DNA sequence does not disrupt expression of the positive selection marker DNA when the promoter sequence is activated within a cell.

91. A DP vector according to claim 90 wherein the third sequence is inserted in the positive selection marker DNA sequence between a promoter sequence and a coding region for the selection marker.

92. A DP vector according to claim 81 wherein the fifth DNA sequence comprises a recombination sequence.

93. A DP vector according to claim 92 wherein the recombination sequence is either 1oxp when the third DNA sequence is 1oxp, or FRT or when the third DNA sequence is FRT.

94. A DP vector according to claim 93 wherein the fifth sequence includes an additional region of DNA and said additional region comprises a PCR primer site or an alternative recombination site.

95. A DP vector according to claim 81 including a further selection marker that is selected from the group consisting of a marker that is the same as the positive selection marker and a marker that is different from the positive selection marker.

96. A DP vector according to claim 95 wherein the further selection marker is flanked by site specific recombination sequences which are under the influence of recombinases.

97. A DP vector according to claim 96 wherein the recombination sequence is loxP or FRT.

98. A DP vector according to claim 95 comprising at least two selectable markers wherein one of the markers is a promoter-less marker and the other marker is under the influence of a promoter.

99. A DP vector according to claim 98 wherein the promoter-less marker is a hygromycin resistance marker (Hyg^{r}).

100. A DP vector comprising a targeting construct according to claim 80.

101. A method for enriching for a transformed cell containing a modification in a target DNA sequence in the genome of said cell comprising:
(a) transfecting cells capable of mediating homologous recombination with a DP selection vector said vector comprising a first homologous vector DNA sequence capable of homologous recombination with a first region of said target DNA sequence; a positive selection marker DNA sequence capable of conferring a positive selection characteristic in said cells ; a third sequence that supports DNA recombination in the presence of a site specific recombinase and which is contained within the positive selection marker; a second homologous vector DNA sequence capable of homologous recombination with a second region of said target DNA sequence; and a fourth sequence which directs site specific recombination with the third sequence, but is substantially incapable of homologous recombination with said target DNA sequence, wherein the spatial order of said sequences in said DP vector is: said first homologous vector DNA sequence, said positive selection marker DNA sequence containing the third sequence, said second homologous vector DNA sequence and said fourth sequence; and wherein the vector is capable of modifying said target DNA sequence by homologous recombination of said first homologous vector DNA sequence with said first region of said target sequence and of said second homologous vector DNA sequence with said second region of said target sequence;
(b) selecting for transformed cells in which said DP selection vector has integrated into said target DNA sequence by homologous recombination by sequentially or simultaneously selecting for transformed cells containing the positive selection marker in the presence of the recombinase; and
(c) analysing the DNA of transformed cells surviving the selecting step to identify a cell containing the modification.

102. A method according to claim 101 wherein the selection for transformed cells is mediated by aCre-/oxP recombinase system.

103. A transformed cell prepared by the method according to claim 101.

104. A method of inducing a modification in genome of a cell, said method comprising:
transfecting cells capable of mediating homologous recombination with a DP selection vector said vector comprising a first homologous vector DNA sequence capable of homologous recombination with a first region of said target DNA sequence; a positive selection marker DNA sequence capable of conferring a positive selection characteristic in said cells; a third sequence that supports DNA recombination in the presence of a site specific recombinase and which is contained within the positive selection marker; a second homologous vector DNA sequence capable of homologous recombination with a second region of said target DNA sequence; and a fourth sequence which directs site specific recombination with the third sequence, but is substantially incapable of homologous recombination with said target DNA sequence, wherein the spatial order of said sequences in said DP vector is: said first homologous vector DNA sequence, said positive selection marker DNA sequence containing the third sequence, said second homologous vector DNA sequence and said fourth sequence; wherein the vector is capable of modifying said target DNA sequence by homologous recombination of said first homologous vector DNA sequence with said first region of said target sequence and of said second homologous vector DNA sequence with said second region of said target sequence.

105. A method according to claim 104 wherein the positive selection marker is positioned in an exon of the gene to be disrupted or modified.

106. A method according to claim 104 wherein the cell is selected from the group consisting of a cell derived from a vertebrate, a cell derived from a mammal, a cell derived from a filamentous fungus, and a cell derived from a plant.

107. A method according to claim 106 wherein the cell is a mammalian cell.

108. A method according to claim 107 wherein the cell is selected from the group consisting of an embryonic cell, a neural cell, an epithelial cell, a liver cell, a lung cell, a muscle cell, an endothelial cell, a mesenchymal cell and a bone stem cell.

109. A method according to claim 104 wherein the DP vector is transfected by electroporation or microinjection.

110. A method of producing a transgenic plant or animal having a genome comprising a modification of a target DNA sequence, said method comprising:
transforming a population of embryonic stem cells with a DP vector;
identifying a cell having said genome by selecting for cells containing said DP vector. and analyzing DNA from cells surviving selection for the presence of the modification;
inserting the cell into an embryo;
propagating a plant or animal from the embryo; wherein the DP vector comprises: a first homologous vector DNA sequence capable of homologous recombination with a first region of said target DNA sequence; a positive selection marker DNA sequence capable of conferring a positive selection characteristic in said cells; a third sequence that supports DNA recombination in the presence of a site specific recombinase and which is contained within the positive selection marker; a second homologous vector DNA sequence capable of homologous recombination with a second region of said target DNA sequence; and a fourth sequence which directs site specific recombination with the third sequence, but is substantially incapable of homologous recombination with said target DNA sequence,
wherein the spatial order of said sequences in said DP vector is: said first homologous vector DNA sequence, said positive selection marker DNA sequence containing the third sequence, said second homologous vector DNA sequence and said fourth sequence;
wherein the vector is capable of modifying said target DNA sequence by homologous recombination of said first homologous vector DNA sequence with said first region of said target sequence and of said second homologous vector DNA sequence with said second region of said target sequence.

111. An animal prepared by the method according to claim 110.

112. A plant prepared by the method according to claim 110.


SEQUENCE LISTING (45 sequences)

SEQ ID NO: 1 - DNA, 84 bp, Artificial Sequence
  Oligonucleotide primer Mu1-1
ctgggtaccagatctgaagcggcgcacgaaaaacgcgaaagcgtttcacgataaatgcgaaaacattcaaatatgtatccgctc

SEQ ID NO: 2 - DNA, 66 bp, Artificial Sequence
  Oligonucleotide primer Mu1-2
ctgcccgggataacttcgtataatgtatgctatacgaagttatcctgtctcttgatcgatctttgc

SEQ ID NO: 3 - DNA, 30 bp, Artificial Sequence
  Oligonucleotide primer Mu1-3
ctggtcgacgctaaggaagctaaaatggag

SEQ ID NO: 4 - DNA, 83 bp, Artificial Sequence
  Oligonucleotide primer Mu1-4
ctgaagcttagatctgaagcggcgcacgaaaaacgcgaaagcgtttcacgataaatgcgaaaacgtcaattattacctccacg

SEQ ID NO: 5 - DNA, 86 bp, Artificial Sequence
  Oligonucleotide primer Mu2-1
ctgggtaccagatctgaagcggcgcacgaaaaacgcgaaagcgtttcacgataaatgcgaaaacttctcatgtttgacagcttatc

SEQ ID NO: 6 - DNA, 30 bp, Artificial Sequence
  Oligonucleotide primer Mu2-2
ctgctcgagccgcaagaattgattggctcc

SEQ ID NO: 7 - DNA, 78 bp, Artificial Sequence
  Oligonucleotide primer Mu2-Neo-1
ctgctcgagataacttcgtatagcatacattatacgaagttataggagccgccaccatgattgaacaagatggattgc

SEQ ID NO: 8 - DNA, 82 bp, Artificial Sequence
  Oligonucleotide primer Mu2-Neo-2
ctgtctagatctgaagcggcgcacgaaaaacgcgaaagcgtttcacgataaatgcgaaaacacacaaaaaaccaacacacag

SEQ ID NO: 9 - DNA, 80 bp, Artificial Sequence
  Oligonucleotide primer Mu2-HygGFP-1
ctgctcgagataacttcgtatagcatacattatacgaagttataggagccgccaccatgaaaaagcctgaactcaccgcg

SEQ ID NO: 10 - DNA, 30 bp, Artificial Sequence
  Oligonucleotide primer Mu2-HygGFP-2
ctgagatcttacttgtacagctcgtccatg

SEQ ID NO: 11 - DNA, 85 bp, Artificial Sequence
  Oligonucleotide primer Mu2-geo-1
ctgctcgagataacttcgtatagcatacattatacgaagttataggagccgccaccatggaagatcccgtcgttttacaacgtcg

SEQ ID NO: 12 - DNA, 41 bp, Artificial Sequence
  Oligonucleotide primer GeoSacBglXba
ctgtctagagagagatcttctgagctcgttatcgctatgac

SEQ ID NO: 13 - DNA, 24 bp, Artificial Sequence
  Oligonucleotide primer SacGeo
ctggagctcctgcactggatggtg

SEQ ID NO: 14 - DNA, 30 bp, Artificial Sequence
  Oligonucleotide primer Mu2-geo-2
ctgagatctcagaagaactcgtcaagaagg

SEQ ID NO: 15 - DNA, 29 bp, Artificial Sequence
  Oligonucleotide primer Mu2-polyA1
ctgggatccgagcagacatgataagatac

SEQ ID NO: 16 - DNA, 89 bp, Artificial Sequence
  Oligonucleotide primer Mu2-polyA2
ctgtctagatctgaagcggcgcacgaaaaacgcgaaagcgtttcacgataaatgcgaaaacttaccacatttgtagaggttttacttgc

SEQ ID NO: 17 - DNA, 20 bp, Artificial Sequence
  Oligonucleotide primer Mu1CamEndOutward
cgtggaggtaataattgacg

SEQ ID NO: 18 - DNA, 20 bp, Artificial Sequence
  Oligonucleotide primer HPRTexon4F
cttgcactcactaggcaagc

SEQ ID NO: 19 - DNA, 24 bp, Artificial Sequence
  Oligonucleotide primer HPRTexon5F
ggacccttctgagttctaataagc

SEQ ID NO: 20 - DNA, 21 bp, Artificial Sequence
  Oligonucleotide primer HPRTexon6F
ccactgcttgcttagaaccag

SEQ ID NO: 21 - DNA, 20 bp, Artificial Sequence
  Oligonucleotide primer HPRTexon7-9F
gttgcatttcagtgtgggtg

SEQ ID NO: 22 - DNA, 51 bp, Artificial Sequence
  Oligonucleotide primer Oligo 1
aattgcggccgcataacttcgtatagcatacattatacgaagttatggtac

SEQ ID NO: 23 - DNA, 43 bp, Artificial Sequence
  Oligonucleotide primer Oligo 2
cataacttcgtataatgtatgctatacgaagttatgcggccgc

SEQ ID NO: 24 - DNA, 43 bp, Artificial Sequence
  Oligonucleotide primer Oligo 3
gataacttcgtatagcatacattatacgaagttatggccggcc

SEQ ID NO: 25 - DNA, 51 bp, Artificial Sequence
  Oligonucleotide primer Oligo 4
agctggccggccataacttcgtataatgtatgctatacgaagttatctgca

SEQ ID NO: 26 - DNA, 40 bp, Artificial Sequence
  Oligonucleotide primer OligoNeo1
ctgggatccgccgccaccatgattgaacaagatggattgc

SEQ ID NO: 27 - DNA, 33 bp, Artificial Sequence
  Oligonucleotide primer OligoNeo2
ctgttaattaacacacaaaaaaccaacacacag

SEQ ID NO: 28 - DNA, 28 bp, Artificial Sequence
  Oligonucleotide primer OligoCMVProm1
ctgggatcctcaatattggccattagcc

SEQ ID NO: 29 - DNA, 64 bp, Artificial Sequence
  Oligonucleotide primer OligoCMVProm2
ctgagatctataacttcgtataatgtatgctatacgaagttatgatctgacggttcactaaacg

SEQ ID NO: 30 - DNA, 27 bp, Artificial Sequence
  Oligonucleotide primer OligoPGKProm1
ctgggatcctaccgggtaggggaggcg

SEQ ID NO: 31 - DNA, 64 bp, Artificial Sequence
  Oligonucleotide primer OligoPGKProm2
ctgagatctataacttcgtataatgtatgctatacgaagttatgtcgaaaggcccggagatgag

SEQ ID NO: 32 - DNA, 65 bp, Artificial Sequence
  Oligonucleotide primer PvulLoxHyg
ctgcgatcgataacttcgtatagcatacattatacgaagttatgccgccaccatgaaaaagcctg

SEQ ID NO: 33 - DNA, 31 bp, Artificial Sequence
  Oligonucleotide primer HygPacl
ctgttaattaagatctatagatcatgagtgg

SEQ ID NO: 34 - DNA, 20 bp, Artificial Sequence
  Oligonucleotide primer HPRTexon7-9F
gttgcatttcagtgtgggtg

SEQ ID NO: 35 - DNA, 20 bp, Artificial Sequence
  Oligonucleotide primer HPRTexon7-9R
aggctgcctacaggctcata

SEQ ID NO: 36 - DNA, 47 bp, Artificial Sequence
  Oligonucleotide primer Kpn-geo-1
ctgggtaccgccgccaccatggaagatcccgtcgttttacaacgtcg

SEQ ID NO: 37 - DNA, 41 bp, Artificial Sequence; identical to SEQ ID NO: 12
  Oligonucleotide primer GeoSacBglXba

SEQ ID NO: 38 - DNA, 26 bp, Artificial Sequence
  Oligonucleotide primer Kpn-PGK-1
ctgggtaccaccgggtaggggaggcg

SEQ ID NO: 39 - DNA, 30 bp, Artificial Sequence
  Oligonucleotide primer MuEndEcoSwaPGK-2
ctgggtaccgtcgaaaggcccggagatgag

SEQ ID NO: 40 - DNA, 29 bp, Artificial Sequence
  Oligonucleotide primer Mu2-polyA1
ctgggatccgagcagacatgataagatac

SEQ ID NO: 41 - DNA, 43 bp, Artificial Sequence
  Oligonucleotide primer PolyA-R
ctgagatctggtaccttaccacatttgtagaggttttacttgc

SEQ ID NO: 42 - DNA, 20 bp, Artificial Sequence
  Oligonucleotide primer HPRTSouthern1
gtactctgtagtccaggctg

SEQ ID NO: 43 - DNA, 20 bp, Artificial Sequence
  Oligonucleotide primer HPRTSouthern2
caagtctttcagtcctgcag

SEQ ID NO: 44 - DNA, 20 bp, Artificial Sequence
  Oligonucleotide primer HPRTSouthern3
gaatagtctaaagcgctcag

SEQ ID NO: 45 - DNA, 22 bp, Artificial Sequence
  Oligonucleotide primer HPRTSouthern4
gctaagagaaagccatgttctc